(19) United States
(12) Patent Application Publication
(10) Pub. No.: US 2023/0273180 A1
(43) Pub. Date: Aug. 31, 2023

(54) SYSTEMS AND METHODS FOR AUTOMATED, REAL-TIME ANALYSIS AND OPTIMIZATION OF FORMATION-TESTER MEASUREMENTS

(71) Applicant: Board of Regents, the University of Texas System, Austin, TX (US)

(72) Inventors: Carlos TORRES-VERDIN, Austin, TX (US); Colin SCHROEDER, Austin, TX (US); Camilo GELVEZ, Austin, TX (US)

(21) Appl. No.: 18/019,205

(22) PCT Filed: Jul. 30, 2021
(86) PCT No.: PCT/US2021/043880

§ 371 Date: Feb. 1, 2023

Related U.S. Application Data
(60) Provisional application No. 63/061,671, filed on Aug. 5, 2020.

(51) Int. Cl. G01N 33/28; G01N 33/18; E21B 49/08

(52) U.S. Cl. CPC G01N 33/2823; G01N 33/1833; E21B 49/08

(57) ABSTRACT

Described herein are methods and systems, and techniques relating to hydrocarbon-bearing formation testing and, particularly, to estimating a formation condition. The disclosed methods, systems, and techniques allow for improved prediction of the formation condition and cleanout of the formation following well drilling. In some cases, the disclosed methods, systems, and techniques include using a formation testing tool to obtain a sampled fluid from a formation according to a set of sampling parameters and using the formation testing tool to analyze the sampled fluid to identify a set of fluid parameters for the sampled fluid. A numerical model may be used to determine a formation condition with inputs including the sampling parameters and the fluid parameters.

### CROSS-REFERENCE TO RELATED APPLICATIONS

This application claims the benefit of and priority to U.S. Provisional Application No. 63/061,671, filed on Aug. 5, 2020, which is hereby incorporated by reference in its entirety.

### FIELD

This application is in the field of subterranean formation evaluation. This application relates generally to systems, methods, and techniques for characterizing formation contamination and conditions and improving prediction methods for oil producing wells.

### BACKGROUND

Well drilling operations introduce contamination into an oil-bearing rock formation that must be cleaned before clean samples of oil or water can be obtained. For example, drilling mud used to lubricate and cool the drilling bit used during drilling of well bores can infiltrate a formation during drilling, contaminating the region of the formation surrounding the well bore. Drilling mud and other contaminants must be cleaned out of the formation before oil can be sampled. In some cases, cleaning is done by pumping out fluid until the fluid produced by the well is clean enough for a particular use. The time required for cleaning, and thus the expense of the cleanout, depends in part on rock formation properties and fluid properties in the region around the well bore, as well as many other factors, including borehole conditions, mud properties, pump out rate, etc.

One step in the process between well drilling and completion is formation testing. Formation testing provides characteristic information about a region in proximity to the borehole (as opposed to well testing, which provides information about a wider region around a well, including drainage area of the well and any boundary effects that may exist within). For example, formation testing permits determination of formation pressures at zones of interest and fluid type identification. Formation testing also allows for identification of zones in hydraulic communication or isolation with the borehole, for collection of representative formation fluid samples, and for estimation of fluid mobility.

In some systems, a testing tool is lowered into the well bore to the depth of the oil-bearing formation, where it collects fluid samples for characterization during a formation test according to a pre-programmed routine. The testing tool may communicate results to the surface via the cable holding it in position or via mud-pulse telemetry. The tool may also store the results locally until it is raised after a period of time for the information to be transferred to a computer on the surface. The testing process may require several days to complete, depending on formation properties. In some cases, testing is done during drilling, through what is referred to as logging while drilling (LWD) or testing while drilling (TWD). In such systems, the drill string itself can include a formation testing tool.

### SUMMARY

Various techniques are described herein for automated evaluation and estimation of rock formation conditions in hydrocarbon-bearing formations, employing numerical models to predict formation conditions. The numerical models may include convolutional neural networks implementing deep learning, whereby numerical models that are pre-trained to predict a formation condition using data collected from prior formation testing of other wells, data from simulations of measurements performed with various formation conditions, and in situ measurements collected by a testing tool. For example, the simulations may represent the response observed by a testing tool when measurements are performed in a formation under a given set of conditions. The techniques may be implemented by computer systems carried by the testing tool into the wellbore, in communication with sensors also carried by the testing tool, such that the tool implements the techniques autonomously (e.g., through feedback control schemes), or by communication between the testing tool and computer systems on the surface. Advantageously, the computer systems may control the operation of the testing tool to implement one or more experimental regimes to accelerate testing and prediction of formation condition, optimizing performance and accuracy of the testing tool. The techniques may permit formation testing to conclude within a shorter timeframe, thereby reducing costs and minimizing the risk of getting the drill string stuck in the wellbore, for example. The computer systems may communicate formation condition information to users on the surface.

The present techniques overcome challenges because implementation in situ eliminates multiple inefficiencies in the formation testing process. For example, communicating information between the testing tool and the surface via mud-pulse telemetry can be slow, have significantly limited bandwidth, and be prone to errors or disruptions. Furthermore, some existing techniques rely on manual control in response to measurements of samples delivered from the formation to the surface, over which distance the samples change chemical composition for reasons including pressure and temperature changes. For example, dissolved gases can come out of solution after being brought to the surface, which can change the physical and chemical properties of the fluid and can bias characterization. As another example, the disclosed techniques may provide refined estimations of the time and/or pump out volume required to clean up the formation in the region around the well bore. Improvement of estimation accuracy and providing an accurate estimation sooner in the testing procedure can reduce costs and time associated with obtaining a suitable sample. In some cases, production equipment and other resources may be inefficiently allocated depending on how representative the acquired samples are of the true formation fluid. For example, if the acquired samples are contaminated, modeling performed using properties measured on those contaminated samples may be inaccurate, causing production equipment and surface facilities to be improperly designed or allocated, which can impart delays and associated costs.

In a first aspect, methods for estimating a formation condition are disclosed herein. In an example, a method of this aspect includes using a formation testing tool to obtain a sampled fluid from a formation according to a set of sampling parameters, using the formation testing tool to analyze the sampled fluid to identify a set of fluid parameters for the sampled fluid; and using a numerical model to determine a formation condition. Inputs for the numerical model may include the set of sampling parameters and the set of fluid parameters. Sampling of the fluid and determination of fluid parameters may occur on a continuous basis. Optionally, a method of this aspect may further include using the numerical model to generate an updated set of sampling parameters, using the formation testing tool to obtain additional sampled fluid from the formation according to the updated set of sampling parameters, using the formation testing tool to analyze the additional sampled fluid to identify an updated set of fluid parameters for the additional sampled fluid, and using the numerical model to generate an updated formation condition.

Inputs for the numerical model may be in any suitable form. Examples include, but are not limited to, the updated set of sampling parameters and the updated set of fluid parameters. Optionally, inputs for the numerical model may further include one or more of historical fluid parameters for fluid sampled from the formation, simulated fluid parameters for fluid sampled from the formation, historical fluid parameters for fluid sampled from a different formation, and simulated fluid parameters for fluid sampled from the different formation.

Similarly, the set of sampling parameters may include sampling conditions associated with obtaining the sampled fluid. For example, the set of sampling parameters may include a drawdown rate used for sampling fluid from the formation, a drawdown pressure used for sampling fluid from the formation, an injection rate for injecting fluid from the formation testing tool into the formation during sampling, a buildup pressure measured after sealing the testing tool, or a characteristic dimension of the formation testing tool. Optionally, the set of sampling parameters further may include a pulse sequence, the pulse sequence including one or more modifications to the drawdown rate, the drawdown pressure, the injection rate, or the buildup pressure in an ordered sequence during sampling fluid from the formation.

It will be appreciated that the set of fluid parameters for the sampled fluid may include analytical results associated with evaluating the sampled fluid. Optionally, the set of fluid parameters for the sampled fluid may include at least one of a mass density for the sampled fluid, a fluid viscosity for the sampled fluid, a fluid resistivity for the sampled fluid, a formation pressure, an estimated formation pressure, an optical density for the sampled fluid, a level of contamination for the sampled fluid, a speed of sound in the sampled fluid, a gas-to-liquid ratio for the sampled fluid, a composition of the sample fluid, or a formation volume factor for the sampled fluid. In some examples, the fluid parameters may be determined as a function of time or as a function of another parameter, such as a pumpout volume.

Advantageously, the formation condition may provide information about the condition of a test well. For example, the formation condition may include one or more of a predicted contamination for additional fluid sampled from the formation as a function of time or pumpout volume, a predicted time at which additional fluid sampled from the formation contains a target amount or less of contamination, a predicted pumpout volume at which additional fluid sampled from the formation contains a target amount or less of contamination, or a predicted lowest level of contamination for additional fluid sampled from the formation. Optionally, a method of this aspect may further include generating a notification providing the formation condition. Optionally, the notification may include one or more of an indication of a predicted lowest level of contamination for additional fluid sampled from the formation, or a predicted duration until additional fluid sampled from the formation contains a target amount or less of contamination. Generating the notification may include communicating the notification to a user device. The numerical model may optionally further generate predicted formation properties that may include one or more of a formation porosity, a formation permeability, a permeability anisotropy, a formation pressure, a formation relative permeability, a formation capillary pressure, a formation water saturation, a formation residual saturation, a formation phase and total mobility, or a formation height.

Various numerical models may be used to generate the formation condition. In some examples, the numerical model evaluates the formation condition by computing a derivative (e.g., a time derivative or a pumpout volume derivative) of one or more fluid parameters of the set of fluid parameters. In some examples, the numerical model evaluates the formation condition by decomposing one or more fluid parameters of the set of fluid parameters as a sum of a plurality of exponentials. Optionally, the numerical model evaluates the formation condition by computing a fluid contamination derivative or a reciprocal contamination derivative. Optionally, the numerical model evaluates the formation condition by decomposing one or more fluid parameters of the set of fluid parameters as a sum of three or more exponentials. Optionally, the numerical model evaluates the formation condition by decomposing the fluid contamination cleanup decay and/or the pressure buildup as a summation of exponentials. In some examples, the formation condition is a time at which a fluid contamination level for fluid from the formation falls or is predicted to fall below a threshold level, the set of fluid parameters includes a contamination level for the sampled fluid, and the numerical model evaluates a time at which a fluid contamination level for fluid from the formation falls or is predicted to fall below a threshold level by decomposing measured fluid contamination levels for the sampled fluid as a sum of a plurality of exponentials.

In another aspect, a formation testing system is described. The formation testing system may include a formation testing tool that may include one or more sampling systems for obtaining a sampled fluid from a formation and additional elements by which the formation testing system may perform a method described here. For example, the formation testing system may be configured with components including, but not limited to, one or more sensors for analyzing the sampled fluid, one or more processors in communication with the one or more sampling systems and the one or more sensors, and a non-transitory computer readable storage medium in communication with the one or more processors, the non-transitory computer readable storage medium containing instructions that, when executed by the one or more processors, cause the one or more processors to perform operations of methods described herein. Optionally, the formation testing system may also perform operations including, but not limited to, one or more of the examples and optional aspects of the disclosed methods.

In another aspect, a computer program product is described. The computer program product may include a non-transitory computer-readable storage medium storing computer-executable instructions that, when executed by one or more processors, cause the one or more processors to perform a method described herein. Optionally the computer program product may also perform operations including, but not limited to, one or more of the examples and optional aspects of the disclosed methods.

Without wishing to be bound by any particular theory, there can be discussion herein of beliefs or understandings of underlying principles relating to the invention. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1 provides a schematic illustration of a hydrocarbon bearing formation and a system for determining a condition of the hydrocarbon bearing formation.

FIG. 2 provides an overview of an example formation condition determination method.

FIG. 3A provides a plot showing simulation results of a sampled fluid contamination fraction as a function of time for a single phase flow.

FIG. 3B provides a plot showing simulation results of a sampled fluid contamination fraction as a function of time for a multiphase flow.

FIG. 4A provides a plot showing simulation results of a sampled fluid reciprocal contamination derivative (RCD) fraction as a function of time for a single-phase flow.

FIG. 4B provides a plot showing simulation results of a sampled fluid RCD fraction as a function of time for a multiphase flow.

FIG. 5A provides a plot showing simulation results of a sampled fluid RCD fraction as a function of time and formation thickness for a single-phase flow.

FIG. 5B provides a plot showing simulation results of a sampled fluid RCD fraction as a function of time and formation thickness for a multiphase flow.

FIG. 6A provides a plot showing simulation results of a sampled fluid RCD fraction as a function of time and thinly-lamination thickness for a single-phase flow.

FIG. 6B provides a plot showing simulation results of a sampled fluid RCD fraction as a function of time and thinly-lamination thickness for a multiphase flow.

FIG. 7A provides a plot showing simulation results of a sampled fluid RCD fraction as a function of time and geological fault position for a single-phase flow.

FIG. 7B provides a plot showing simulation results of a sampled fluid RCD fraction as a function of time and geological fault position for a multiphase flow.

FIG. 8A provides a plot showing simulation results of a sampled fluid RCD fraction as a function of time for a single-phase flow.

FIG. 8B provides a plot showing simulation results of a sampled fluid RCD fraction as a function of time for a multiphase flow.

FIG. 9A provides a plot showing simulation results of a sampled fluid RCD fraction as a function of time and mud filtrate invasion depth for a single-phase flow.

FIG. 9B provides a plot showing simulation results of a sampled fluid RCD fraction as a function of time and mud filtrate invasion depth for a multiphase flow.

FIG. 10A provides a plot showing simulation results of a sampled fluid RCD fraction as a function of time for a single-phase flow.

FIG. 10B provides a plot showing simulation results of a sampled fluid RCD fraction as a function of time for a multiphase flow.

FIG. 11A provides a plot showing simulation results of a sampled fluid RCD fraction as a function of time and permeability anisotropy for a single-phase flow.

FIG. 11B provides a plot showing simulation results of a sampled fluid RCD fraction as a function of time and permeability anisotropy for a multiphase flow.

FIG. 12A provides a plot showing simulation results of a sampled fluid RCD fraction as a function of time for a single-phase flow.

FIG. 12B provides a plot showing simulation results of a sampled fluid RCD fraction as a function of time for a multiphase flow.

FIG. 13 provides a plot showing logging while drilling (LWD) experimental measurements of a sampled fluid contamination fraction as a function of time.

FIG. 14 provides a plot showing LWD experimental measurements of a sampled fluid RCD fraction as a function of time.

FIG. 15A provides a plot showing LWD experimental measurements of a sampled fluid contamination fraction as a function of time.

FIG. 15B provides a plot showing LWD experimental measurements of a sampled fluid contamination fraction as a function of time.

FIG. 16A provides a plot showing LWD experimental measurements of a sampled fluid RCD fraction as a function of time.

FIG. 16B provides a plot showing LWD experimental measurements of a sampled fluid RCD fraction as a function of time.

FIG. 17 provides an illustration representing a sumerical simulation model showing a grid refinement and a top view of a near-wellbore zone during fluid a cleanup simulation.

FIG. 18 provides log-log plot of fluid contamination and fluid contamination derivative (FCD).

FIG. 19A and FIG. 19B provide log-log plots of fluid contamination and FCD for a multiphase flow case.

FIG. 20 provides a log-log plot showing a sensitivity analysis for noise reduction and over smoothing evaluation in the application of the FCD.

FIG. 21A and FIG. 21B provide log-log plots of fluid contamination and FCD for a radial boundary case.

FIG. 22A and FIG. 22B provide log-log plots of fluid contamination and FCD for a vertical boundary case.

FIG. 23A and FIG. 23B provide log-log plots of fluid contamination and FCD for thinly-lamination case.

FIG. 24A and FIG. 24B provide log-log plots of fluid contamination and FCD for a mud-filtrate invasion radius case.

FIG. 25A and FIG. 25B provide log-log plots of fluid contamination and FCD for a reservoir properties case.

FIG. 26A and FIG. 26B provide log-log plots of fluid contamination and FCD for a permeability anisotropy case.

FIG. 27A and FIG. 27B provide log-log plots of fluid contamination and FCD for a Gaussian noise case.

FIG. 28A and FIG. 28B provide plots comparing RCD and pump-out volume (PV) data for a base case and a reservoir limit case.

FIG. 29A and FIG. 29B provide plots comparing RCD and PV data for a base case and a near wellbore features case.

FIG. 30A and FIG. 30B provide plots of real time contamination target estimation data for a base case cleanup curve, and PV distribution.

FIG. 31 is a diagram illustrating an example architecture for implementing an automated formation condition estimation technique, in accordance with at least one embodiment.

### DETAILED DESCRIPTION

Described herein are methods, systems, and techniques relating to evaluating conditions and properties of fluid bearing rock formations (e.g., oil-, water-, or gas-bearing rock formations) and, particularly, involving automated in situ testing techniques implementing model simulations. The disclosed methods, systems, and techniques allow for determination of an accurate amount of time needed to obtain a suitably clean sample from a fluid bearing rock formation using a formation testing tool, such as by using real-time downhole measurements in a time estimation model. The time estimation model can employ previous measurements from the same or other wells and known or modeled information about the formation. In some cases, the disclosed methods, systems, and techniques can also improve or reduce the amount of time needed for obtaining a suitably clean sample by altering the operational parameters of the testing tool in response to the downhole measurements or modeling results. Both the time estimation model and operational parameters can be adjusted according to outputs from a numerical model, for example models employing physics-guided neural networks or time-series machine learning, that can use the real-time downhole measurements or previous measurements. Advantageously, the sensors and components of formation testing tools may be suitable for carrying out the disclosed techniques, but their operation can be optimized to improve sampling and reduce the time needed for obtaining a clean sample. For example, pump pulse sequences, drawdown or injection rate or sequence, sampling pressure, testing tool sampling orifice size or probe shape, or the like can be optimized. The numerical model can also allow for identification of issues that may occur during sampling, such as tool failure or sampling port plugging, allowing mitigation of these issues, such as by modifying sampling parameters to prevent further plugging.

In general the terms and phrases used herein have their art-recognized meaning, which can be found by reference to standard texts, journal references and contexts known to those skilled in the art. The following definitions are provided to clarify their specific use in the context of the invention.

“Fluid bearing formation” refers to any subterranean rock formation that contains liquid or gaseous fluids or mixtures of liquid and gaseous fluids (also referred to as a “formation”). A specific example of a fluid bearing formation is an oil bearing formation, which may contain liquid and/or gaseous hydrocarbons. A formation may include any type of mineral structure or composition associated with petroleum production from land-based wells and/or undersea wells, for example.

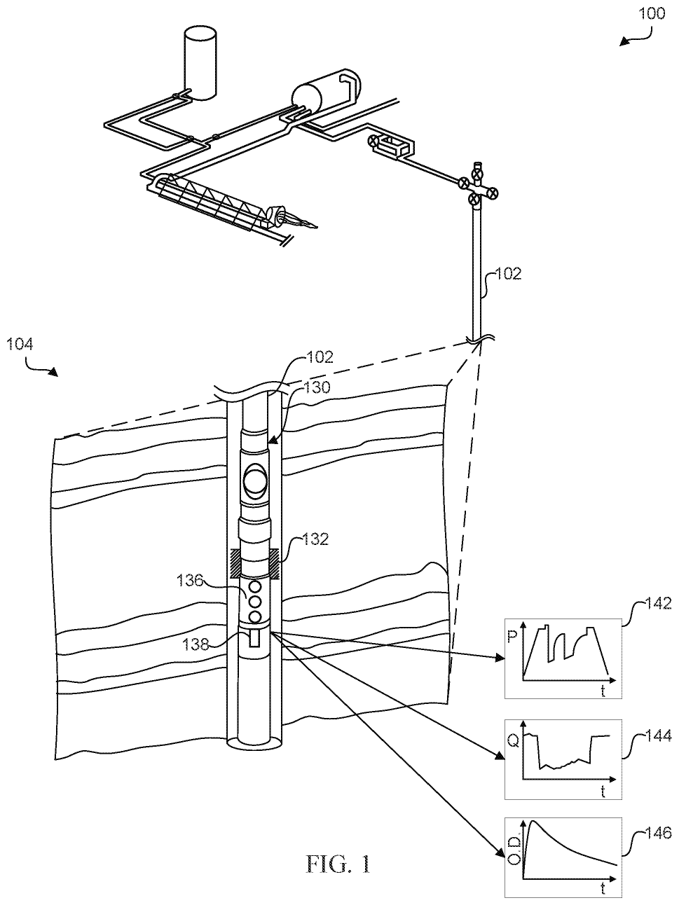

“Testing tool” refers to any downhole testing or sample collection apparatus as described in more detail in reference to FIG. 1 (also referred to as a “tool”). This may include but is not limited to downhole tools used for wireline formation testing, testing while drilling and logging while drilling tools, or the like. One specific example of a testing tool may be a modular formation dynamics tester (MDT) tool.

“Buildup” refers to a formation testing regime whereby a quantity of fluid is sampled from a formation, after which either the well bore is sealed or the testing tool is sealed. The tool measures pressure buildup subsequent to sealing from which related formation and fluid properties may be estimated, including permeability, fluid viscosity, pressure differential, hole volume, and zone thickness.

“Drawdown” refers to measurements of fluid pressure in the formation during sampling, which may be related to fluid and formation properties including but not limited to fluid viscosity and formation permeability. For example, the pressure may progressively drop during fluid extraction, indicating a low permeability or a high viscosity. Additionally, pressure drop may indicate that the region of the formation surrounding the wellbore may be damaged.

“Open hole exposure” refers to exposure of the fluid bearing formation to the well bore. This is in contrast to a cased well bore where the formation is isolated from the wellbore by a casing (also referred to as a “cased” hole).

“Pumpout” refers to removal of fluids from the fluid bearing formation and discharge to the wellbore. In some cases, the terms “pumpout” and “cleanup” are used interchangeably. In some cases, fluids removed from the fluid bearing formation may be analyzed prior to discharge to the wellbore.

“Contamination” refers to fluids associated with a drilling process that permeate a formation from a wellbore but which are not representative of the fluids present in the formation prior to drilling. In some cases, for example, drilling mud can enter regions of a formation adjacent to a wellbore during a drilling process and can be considered contamination.

“Formation volume factor” refers to a ratio of a volume of a quantity of a fluid at formation temperature and pressure conditions to the volume of the quantity of the fluid at standard temperature and pressure conditions.

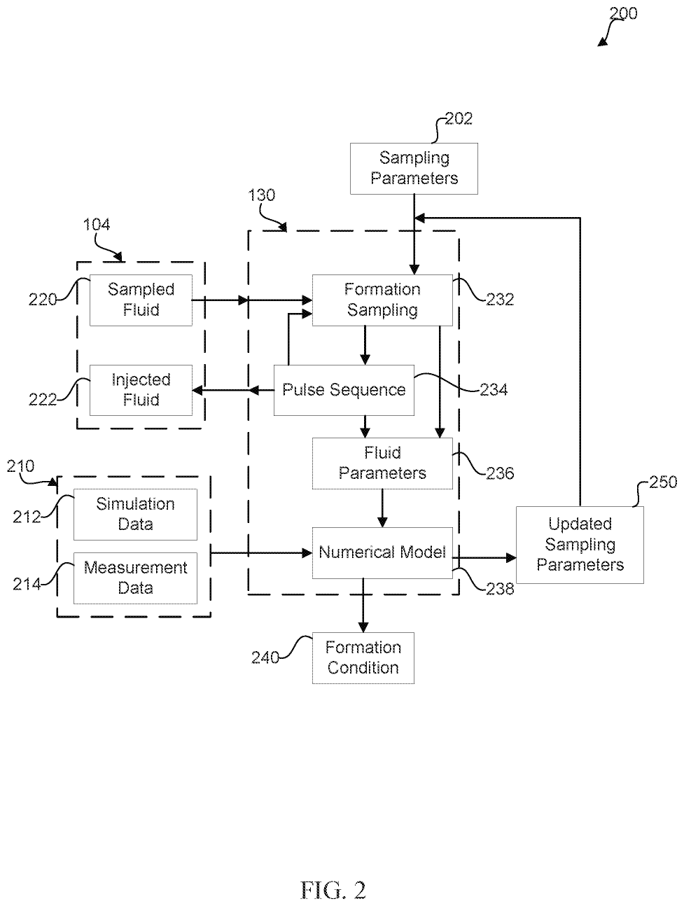

FIG. 1 provides a schematic illustration of a hydrocarbon bearing formation 104 and a system 100 for determining a condition of the hydrocarbon bearing formation 104. In some cases, the system 100 includes a well 102 drilled into the formation 104. The formation 104 may be located beneath the surface of the earth, either beneath a continent or under the sea floor, at depths up to several kilometers, for example. Various systems may be present on the surface above the formation 104, such as on land or on a floating structure. During testing, the well 102 may produce fluids including but not limited to a liquid hydrocarbon, a gas (e.g., a gaseous hydrocarbon or other gas) dissolved in the liquid phase, an aqueous slurry (e.g., drilling mud), a non-aqueous slurry, a treatment fluid, water, or the like, and a gas phase including but not limited to gaseous hydrocarbons, carbon dioxide, or the like. In some cases, the samples are drawn from the formation 104 by a testing tool 130 positioned in the well 102 at the location of the fluid bearing layers of the formation 104. In some cases, the testing tool 130 is a test while drilling tool and/or a logging-while-drilling tool. The testing tool 130 may include one or more components designed to implement one or more testing methods, including but not limited to drawdown tests, buildup tests, pulse sequence tests, or fluid sampling and characterization, as described in more detail in reference to FIG. 2. An example testing tool may be a modular formation dynamics tester (“MDT”). In some cases, the testing tool 130 includes a packer 132, which may be inflatable or otherwise extensible, to fill the volume between the tool and the walls of the well 102, which may be cased. In this way, deploying the packer 132 seals the well 102 at the position of the formation 104 and prevents fluids originating outside to the formation 104 from entering the testing tool 130. In some cases, the testing tool 130 includes more than one packer 132, for example to seal the well 102 both above and below the testing tool 130.

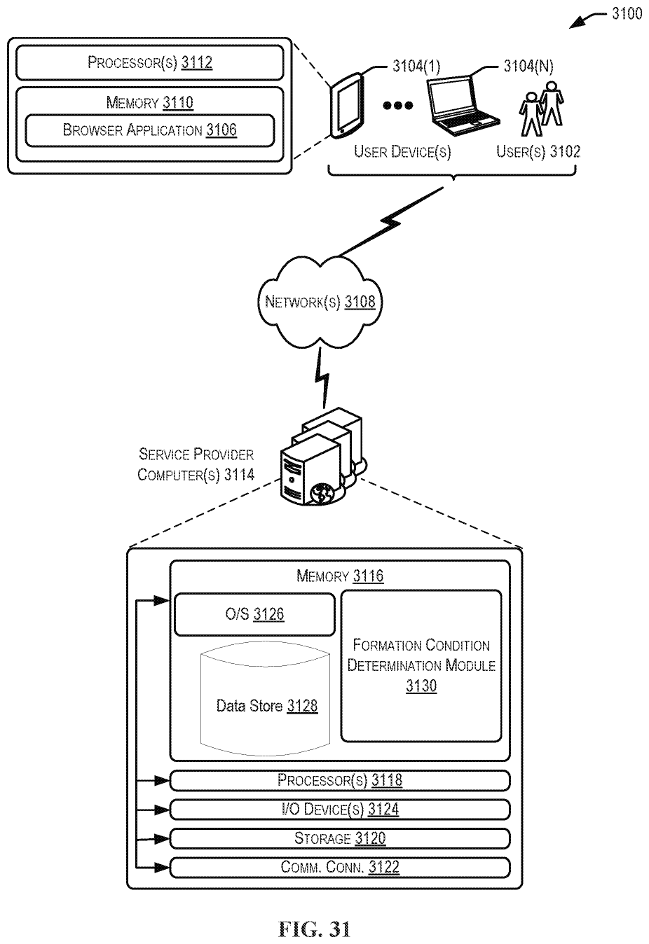

In some cases, the testing tool includes a sampling apparatus 136, designed with one or more orifices of variable characteristic dimension, as described in more detail in reference to FIG. 2. In some cases, the testing tool 130 may include extensible positioners (not shown) to position the sampling apparatus 136 directly against the formation 104 for direct fluid sampling. In some embodiments, the testing tool includes multiple testing and probe assemblies 138, including but not limited to pressure gauges, vertical permeability probes, horizontal permeability probes, sink probes, optical density probes, resistivity probes, and the like. In some cases, the testing tool 130 includes onboard electronics including but not limited to programmable controllers, transitory and/or non-transitory memory units, one or more processors, a communications unit configured to communicate information to a user device on the surface, and the like as described in more detail in reference to FIG. 31. In some cases, the testing tool 130 may include one or more components configured to implement a pulse sequence, including but not limited to pumps or piston systems, the use of which is described in more detail in reference to FIG. 2.

In some cases, the testing tool 130 uses the sampling apparatus 136 and the probe assemblies 138 to collect data about the formation 104 and the fluid sampled therefrom including, but not limited to pressure data 142, fluid flowrate data 144, optical density data 146, or other fluid property data, such as fluid viscosity, mass density, resistivity, or the like. In some cases, the data may form a part of the inputs to models executed by the testing tool 130 or a related system as described in more detail in reference to FIG. 2. In some cases, the testing tool 130 may operate autonomously according to software stored in the memory units to determine a condition of the formation 104, one or more parameters of the fluid in the formation 104, a level of contamination in the formation 104, among other data as described in more detail in reference to FIG. 2.

FIG. 2 provides an overview of an example formation condition determination method 200. In some cases, the method 200 includes the testing tool 130 receiving one or more sampling parameters 202. The sampling parameters 202 may be stored in non-transitory memory carried by the testing tool 130 and/or received by the testing tool 130 from a user device on the surface. In some cases, the testing tool 130 implements the sampling parameters 202 to draw sampled fluid 220 from the formation 104 during formation sampling 232. In some cases, the testing tool 130 can detect occlusion of the sampling apparatus (e.g., the sampling apparatus 136 of FIG. 1) by solids, slurry, and/or viscous components of the sampled fluid 220, such as by monitoring pressure sensor measurements. In some cases, the testing tool 130 may modify one or more sampling parameters 202 to compensate for the occlusion. As an example, the testing tool 130 may send injected fluid 222 into the formation to wash out the occlusion. In some cases, the testing tool 130 may modify the characteristic dimension of the sampling apparatus to implement the formation sampling 232, such as by switching to a different sampling probe. In some cases, the testing tool may modify the pumpout rate to compensate for the occlusion. As described in reference to FIG. 1, the method 200 may include a pulse sequence 234, which may involve sending injected fluid 222 into the formation 104 under controlled conditions. For example, the pulse sequence 234 may include multiple phases of drawing sampled fluid 220 from the formation over a period of time (also referred to as a drawdown test), followed by sending injected fluid 222 into the formation 104. In some cases, the pulse sequence 234 may include a buildup pressure measurement, whereby the testing tool 130 measures the buildup of pressure in the formation 104 over time after sealing the sampling apparatus of the testing tool 130. In some cases, the pulse sequence 234 may permit the testing tool 130 to measure additional properties of the formation 104 and/or to measure properties of the formation 104 more accurately, as described in more detail in reference to Example 1, below.

In some cases, the testing tool 130 determines one or more fluid parameters 236 from the data collected from the formation sampling 232 and/or pulse sequence 234 operations. The fluid parameters may include, but are not limited to, a mass density, a fluid viscosity, a fluid resistivity, a formation pressure, an estimated formation pressure, an optical density (“OD”), a level of contamination, or the like. In turn, the fluid parameters 236 may form part of the inputs to a numerical model 238 implemented by the testing tool 130. Input data 210 may also be provided to the numerical model to improve and/or refine model outputs including but not limited to simulation data 212 and measurement data 214. Simulation data 212 may include data generated by simulations for formation sampling and pulse sequence outputs based on analytical methods, physics-based models, or numerical methods (e.g., based on neural network models trained on empirical data collected from previous formation tests). In some cases, the numerical model 238 may generate one or more outputs, including but not limited to the formation condition 240, time values corresponding to one or more industrially relevant parameters, target testing values, and the like. The numerical model may be trained using previously obtained data (e.g., simulation data and/or measurement data for other or related formations) and/or known outputs (e.g., for other or related formations) to predict output parameters for a formation under test. In some examples, the numerical model 238 may generate a predicted time (e.g., a date and time of day) at which the sampled fluid 220 will contain a given concentration or less of one or more contaminants or a total amount of time until the sampled fluid 220 will contain a given concentration or less of one or more contaminants.

For example, if a sampled fluid 220 contains drilling mud filtrate above a threshold concentration such that the OD of the fluid is above a corresponding threshold value, the model may determine, based on fluid parameters 236 including OD measurements, a time at which the drilling mud concentration will fall below the threshold concentration, based at least in part on a cleanup operation and the formation condition 240. In some cases, the numerical model 238 may generate a predicted pumpout volume after which the sampled fluid 220 will contain a given concentration or less of one or more contaminants. In some cases, the numerical model may determine a duration of time until the sampled fluid 220 drawn from the formation 104 will be sufficiently free of one or more contaminants. Such calculations may depend on the sampling parameters 202, and as such the numerical model may determine updated sampling parameters 250 to permit the testing tool 130 to determine additional outputs and/or to generate outputs more rapidly or more accurately. For example, the model may generate updated sampling parameters 250 to draw additional sampled fluid 220 when the formation condition 240 indicates occlusion or high contamination. As a specific example, the model may indicate a different pumpout rate should be used. In some cases, the updated sampling parameters 250 may replace the sampling parameters 202, such that the testing tool 130 implements formation sampling 232 or pulse sequence 234 operations only according to the updated sampling parameters 250. In some cases, the updated sampling parameters 250 may include the sampling parameters 202 to the extent that the updated sampling parameters 250 may not include updates to one or more of the sampling parameters 202.

In some cases, the numerical model 238 may be implemented in a convolutional neural network as a machine learning algorithm. In some cases, the algorithm may be a supervised learning model, trained over a period of 1-5 hours or 1-10 days using a classified dataset of formation condition values and fluid parameters and testing regimes. In some cases, the algorithm may be an unsupervised learning model, trained to cluster fluid parameters 236 with input data 210 from the same or other wells and/or simulation data 212 to determine formation properties. Use of a combination of input data 210, which may contain historical data, for example, and simulation data 212, which may contain physics-based simulation results may be advantageous as such a combination can be useful for filling in gaps of historical data to allow for sufficient coverage of the sample space to train an algorithm. For example, the machine learning algorithm may be trained to minimize a loss function with respect to one or more of the formation condition 240 parameters based on the fluid parameters 236 as measured by the testing tool. The loss function may be based on one or more analytical methods including but not limited to the techniques described in more detail in reference to Example 1, below.

The invention may be further understood by the following non-limiting examples. The following examples are not intended to describe a preferred embodiment, but rather examples among many possible examples, being used herein for illustrative purposes.

### Example 1: Optimizing Logging while Drilling Fluid Sampling with a New Transient Approach: The Reciprocal Contamination Derivative

Successful in situ fluid cleanup and sampling operations are commonly driven by a fast and reliable analysis of pressure, rate, and contamination measurements. Currently, techniques such as pressure transient analysis (PTA) and rate transient analysis (RTA) provide important information to quantify reservoir complexity, whereas fluid contamination measurements are overlooked for reservoir characterization purposes. This example describes a technique to relate fluid contamination measurements with reservoir properties by identifying early- and late-time flow regimes in the derivative plots of reciprocal fluid contamination. Among several applications, this new transient analysis method is effective for improving logging-while-drilling (LWD) fluid sampling operations.

Contamination transient analysis (CTA) evaluates transient measurements acquired during mud-filtrate invasion cleanup to infer reservoir geometry. The techniques described in this example apply derivative methods to the reciprocal of the time evolution of fluid contamination to identify flow regimes in cases of water-based mud invading either water- or hydrocarbon-saturated formations. LWD operations are considered under a continuous invasion effect, i.e., the fluid cleanup procedure is performed while mud filtrate continues to invade the formation. This constraint brings about a significant technical challenge for LWD fluid sampling jobs. Alternatively, the techniques described in this example could be integrated with other pressure transient techniques to improve the interpretation of measurements. For example, in a pretest case where the pressure transient does not achieve the radial flow regime, fluid cleanup could provide complementary information about late-time flow regimes to enhance the acquisition of measurements in real time.

The techniques described in this example document synthetic and field examples of applications of a new interpretation method. Seven reservoir cases are simulated to obtain contamination data: (1) homogeneous isotropic reservoir, (2) formation thickness, (3) laminated formations, (4) geological faults, (5) mud-filtrate invasion (6) reservoir properties, and (7) permeability anisotropy. All these cases are compared for single-phase and multiphase flow during LWD fluid sampling operations. Additionally, field case studies are analyzed to highlight the value of the reciprocal contamination derivative (RCD) in real-time operations. Reservoir limits and features such as saturating fluid and depth of invasion are identified in the flow regimes detected with derivative plots of the reciprocal of the contamination. Consequently, LWD cleanup and sampling efficiency could be optimized based on contamination transient analysis by identifying the flow regimes taking place in the reservoir during filtrate cleanup, hence improving the prediction of the time required to acquire non-contaminated fluid samples.

The approach of the reciprocal contamination derivative is an alternative way to optimize fluid cleanup efficiency and to quantify the spatial complexity of the reservoir during real-time LWD operations. In addition, this technique enables the evaluation of reservoir properties in less operational time than PTA without the need of pressure build-up stages, increasing fluid sampling efficiency in terms of quality and time.

Introduction. During real time cleanup operations, change in contamination can be continuously determined to assess fluid sample quality. This is currently the sole use for contamination data despite the great amount of information collected over several minutes of fluid pumpout. Likewise, post-job applications often overlook cleanup measurements. In contrast, pre-test pressure data provide formation pressure measurements, vertical connectivity, and fluid contacts, and pressure transient analysis (PTA) estimates reservoir mobility and vertical boundaries in thin layers. However, both short pre-test duration and small volume of investigation significantly limit PTA techniques in formation testing applications. Techniques described in this example introduce a new transient analysis method based on contamination determination using downhole measurements of various fluid properties, such as optical density, mass density, sound speed, electrical resistivity, etc., to increase formation-testing capabilities in reservoir description and to improve cleanup efficiency in real time.

PTA and rate transient analysis (RTA) concepts, such as pressure derivative and reciprocal of flowrate are useful to develop analogous theories for contamination transient assessment. RTA and PTA both investigate the transient behavior of flowrate and pressure diffusion. RTA evaluates the reciprocal of flowrate due to its mathematical definition and interpretative relation to decline curve analysis. On the other hand, PTA employs pressure derivative methods to identify flow regimes, reservoir geometry and boundaries. Some methods may apply a center-point technique which considerably reduces the effect of noise in the calculation of pressure derivatives. Noise reduction is significant for real-time measurements because the acquired data often include large noise-to-signal ratios and uncertainty related to the physics of the measurements.

The techniques described in this example introduce the concept of contamination transient analysis (CTA), and implement the method of the reciprocal contamination derivative (RCD). CTA studies the transient behavior of mud-filtrate concentration during fluid cleanup. An analytical model exists to represent and predict mud-filtrate cleanup performance at late times. Moreover, fluid cleanup simulations have been performed for diverse formation testing tools and reservoir conditions, and have identified three different flow regimes: (1) short period, (2) intermediate period, and (3) late-time period; also these flow regimes have been defined as intermediate regime and developed flow. In the following sections, these flow regimes are classified as early- and late-time, and the RCD method is described, which enhances flow regimes identification and provides new insights on filtrate contamination measurements applications.

It is clear that the CTA and RCD techniques have the potential to positively influence formation-testing operations when used to quantify reservoir complexity and understand cleanup trends to achieve optimal fluid sampling conditions. Specifically, information about reservoir geometry and boundaries, presence of laminations, invasion depth, and permeability anisotropy are key to optimize fluid pumpout. The techniques described in this example expand the proficiency of formation testing and overcomes the limitations of PTA. The following sections introduce the RCD method, describe several numerical simulation studies, and document two field applications to verify the value of the CTA in reservoir characterization and fluid sampling operations.

Methods. Reciprocal Contamination Derivative Method. This transient technique comprises two fundamental steps based on RTA and PTA concepts. Similar to RTA applications, contamination data are inverted due to the physics of flow diffusion and taking into account the fact that cleanup measurements are driven by the pumping rate. Thus, the reciprocal contamination is defined as the inverse of the contamination fraction (1/C). Subsequently, the derivative of the reciprocal contamination is implemented using a center-point derivative method. Accordingly, merging the reciprocal and the derivative concepts gives rise to the new RCD method, given by

$${( \frac{d ( 1 / C )}{dt} )}_{i} = \frac{\frac{{Δ ( 1 / C )}_{1}}{Δ {t}_{1}} Δ {t}_{2} + \frac{{Δ ( 1 / C )}_{2}}{Δ {t}_{2}} Δ {t}_{1}}{Δ {t}_{1} + Δ {t}_{2}} \qquad ( 1 )$$

where Δ(1/C) is the variation of the reciprocal contamination and Δt is the range in time. In addition, subscripts i, 1 and 2 represent the center point and its locations before and after, respectively.

Numerical Simulation of Reservoir Cases. Numerical simulation is a reliable approach to verify the RCD technique under diverse reservoir conditions. To that end, the techniques described in this example use a compositional numerical algorithm to reproduce water-base mud (WBM) filtrate invasion and fluid sampling in a water- (single-phase flow) and a hydrocarbon-saturated formation (multiphase flow). Both models consist of a cylindrical grid refined in the near-probe and near-wellbore regions to accurately simulate the complexity of fluid flow phenomena taking place in the invaded zone. Table 1 contains the input parameters of these numerical simulation models. Furthermore, history matching of contamination cleanup measurements calibrates the model and validates the simulation results. The construction and validation of numerical models provide quality control for the output data required for testing the contamination transient technique.

TABLE 1
Summary of input properties for numerical simulation models.

| Parameter | Value | Units |
|---|---|---|
| Reservoir thickness | 100 | ft |
| External radius | 400 | ft |
| Wellbore radius | 0.354 | ft |
| Porosity | 0.20 | — |
| Permeability | 80 | md |
| Reservoir pressure | 1890.75 | psia |
| Permeability | 0.06 | — |
| anisotropy (kv/kh) | | |
| Mud-filtrate viscosity | 1.0 | cP |
| Formation fluid | 1.0 | cP |
| viscosity, | | |
| single-phase flow | | |
| Formation fluid | 2.0 | cP |
| viscosity, | | |
| multiphase flow | | |

Once the models are calibrated, seven reservoir simulation cases are constructed to obtain synthetic data to implement the RCD method: (1) Base case: homogeneous isotropic reservoir, (2) formation thickness, (3) thin laminations, (4) geological faults, (5) mud-filtrate invasion, (6) reservoir properties, and (7) permeability anisotropy. A homogeneous isotropic reservoir is a suitable reference for comparison of various reservoir conditions. For instance, simulation models varying formation thickness and placing geological faults (no-flow barriers) at different distances from the wellbore are useful to estimate reservoir limits employing transient analysis. Thin-layered models, on the other hand, reproduce the effects of shale laminations on both fluid cleanup and the contamination derivative method. Likewise, models with variable invasion volume could quantify and solve the uncertainty of invasion depth during single-phase fluid sampling operations. Numerical models with a wide range of porosities, permeabilities, and degrees of anisotropy can be used to quantify the impact of these significant petrophysical properties on the interpretation of the contamination transient technique. Consequently, the simulation of multiple reservoir conditions confirmed the applicability of the RCD method.

LWD Fluid Sampling Cases. Field data validate the reliability and practical applications of the RCD method. Two logging-while-drilling (LWD) fluid sampling cases were described to confirm the RCD results and the CTA theory. LWD provides an ideal application for the RCD method because while-logging measurements pose additional challenges for real-time formation evaluation and fluid sampling optimization. For example, LWD invasion mechanisms vary in comparison to wireline logging due to mud-cake build-up and thickness, invasion time, and openhole exposure. Similarly, operational considerations can limit the effective pumpout time to acquire representative reservoir-fluid-samples. Therefore, LWD fluid sampling requires the application of new techniques, such as the contamination transient approach, to increase the interpreter's ability to identify and overcome the above-mentioned challenges.

For field applications, noise filters are necessary to enable an accurate assessment of contamination measurements and subsequent calculation of the contamination transients via the RCD method. Presence of noise in the measurements is a major concern in derivative approaches, where the calculation of the derivative implicitly enhances the effect of noise. To circumvent this difficulty, noise filters were implemented on the measurements, the reciprocal contamination and the derivative outputs. These three filters calculate the median of the data using independent and adaptable time windows, which automatically adjust their time length according to measurements noise-to-signal-ratio and the stages of fluid cleanup and sampling. The median filter is suitable for the interpretation of contamination data because it eliminates the impact of data outliers commonly encountered during pump-out operations. In addition, the techniques described in this example employ a center-point method to calculate the derivative of the reciprocal of contamination. It is found that the use of multiple median filters and a center-point derivative effectively decrease the effect of noise in the RCD technique.

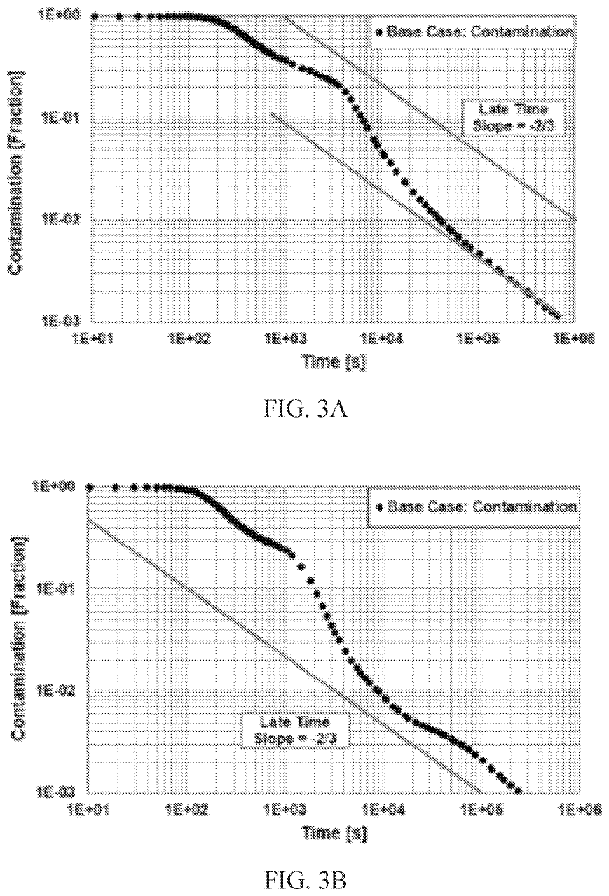

Results. Numerical Simulations. Base Case: Homogeneous Isotropic Reservoir. This case consists of a 100 ft thickness and 400 ft radial extension clean homogeneous-isotropic reservoir with a porosity of 20%, permeability of 80 md, and a constant mud-filtrate invasion radial length of 6 in. FIGS. 3A and 3B show the contamination curve for the base case model for single-phase flow (a) and multiphase flow (b), including the late-time trend in the contamination curve introduced and confirmed as t^{-2/3}.

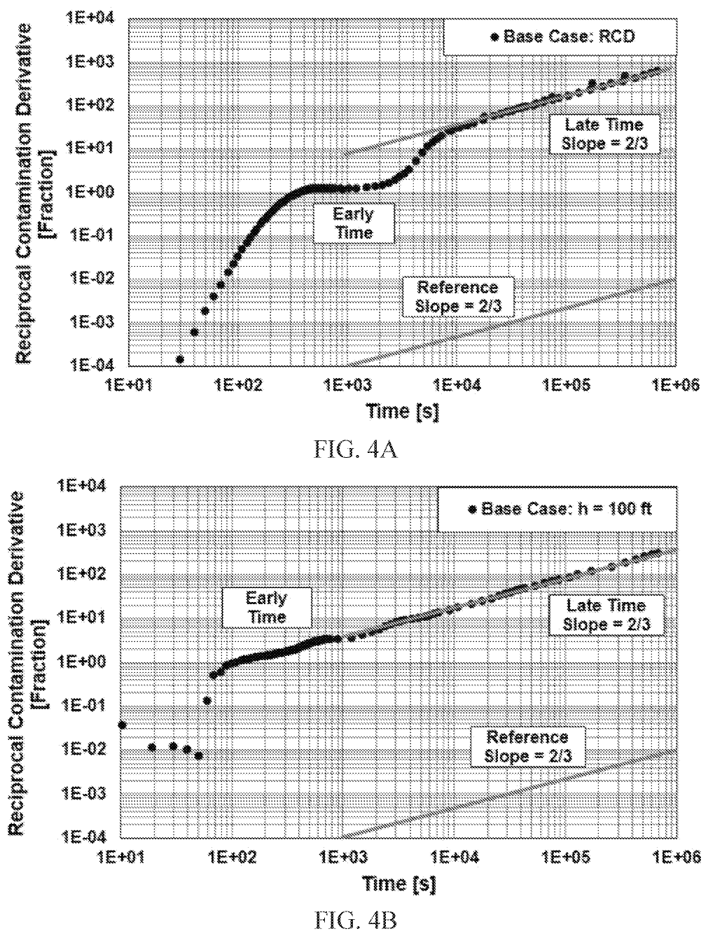

Similarly, FIGS. 4A and 4B illustrate the RCD curve for single-phase flow (a) and multiphase flow (b). For the single-phase flow simulations, the RCD curve exhibits distinct trends for early- and late-time regimes. The early-time regime characterizes for a constant horizontal trend, while the late-time regime, for both flow type simulations, displays a slope of approximately ⅔. Consequently, because of the infinite conditions of the synthetic models, this slope suggests the presence of a spherical flow regime at late times.

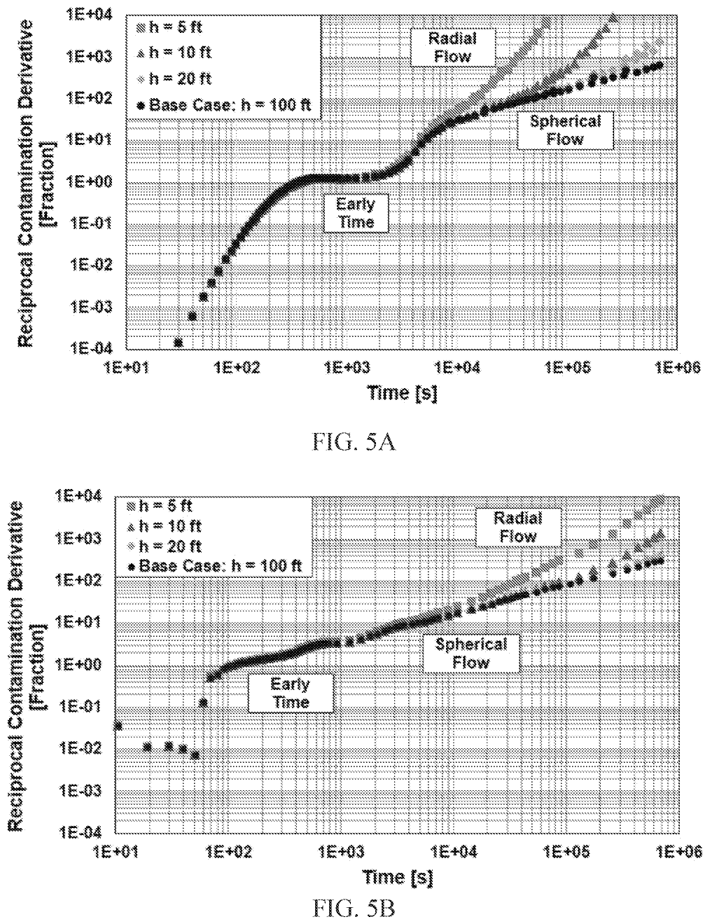

Identification of Reservoir Boundaries. The formation thickness case reproduces the presence of vertical limits at several distances from the formation-testing tool to investigate the effect of reservoir seals in the contamination transient. FIGS. 5A and 5B describe the results for 5 ft, 10 ft and 20 ft reservoir thickness compared to the base case. According to the RCD curves, no observable differences exist during the early-time regime; by contrast, notable differences arise during the late-time regime. All formation thickness curves exhibit a slope higher than the spherical flow slope observed in the base case. As thickness increases, the RCD late-time trend approximates to the spherical flow slope. Thus, simulation results permit the identification of a radial flow regime. This flow regime is attained when the contamination transient reaches the vertical seals of the formation, and the flow regime changes from spherical to radial flow.

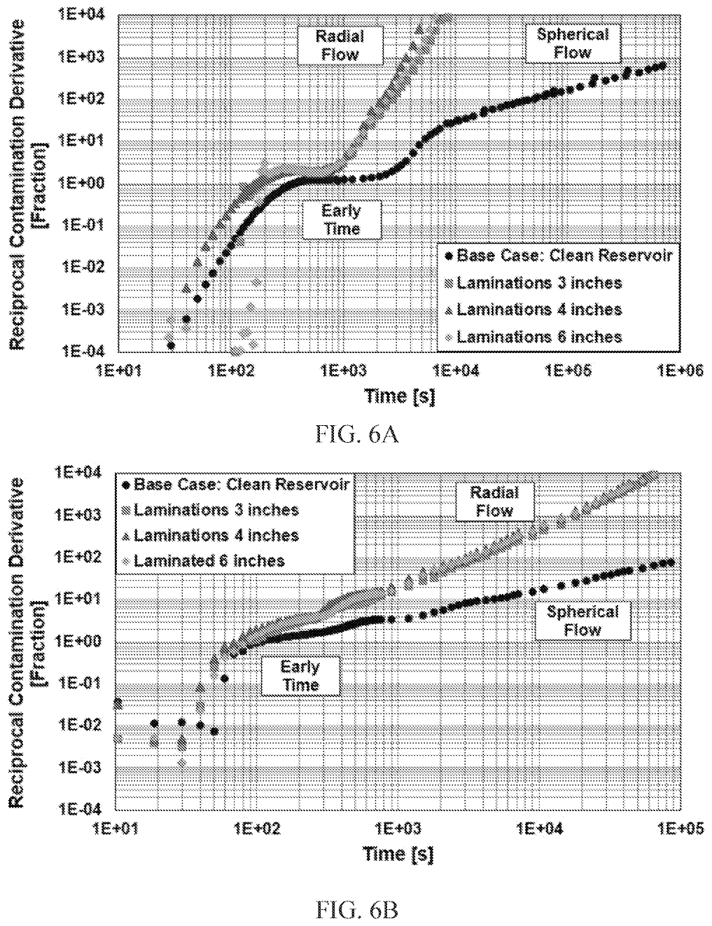

Likewise, the thinly laminated formation case exhibits a radial flow regime with a signature of an increasing slope considerably larger than the slope exhibit by the spherical flow regime. FIGS. 6A and 6B compare the reservoir models of 3 in, 4 in, and 6 in laminations to the base case. As explained in the previous simulation case, the early-time regime trends are equal to those of the homogeneous model. Nevertheless, for the laminated models, the late-time regime exhibits a steep slope similar to that of the formation thickness response, which confirms the effect of vertical boundaries and the existence of the radial flow regime. In addition, the radial flow regime emerges earlier in thin laminations because the contamination transient senses the vertical limits faster in these simulations than in the formation thickness cases. Interestingly, the three laminated reservoir curves almost overlap at late times, allowing easier detection of shale laminations via the RCD application, independently of their thickness.

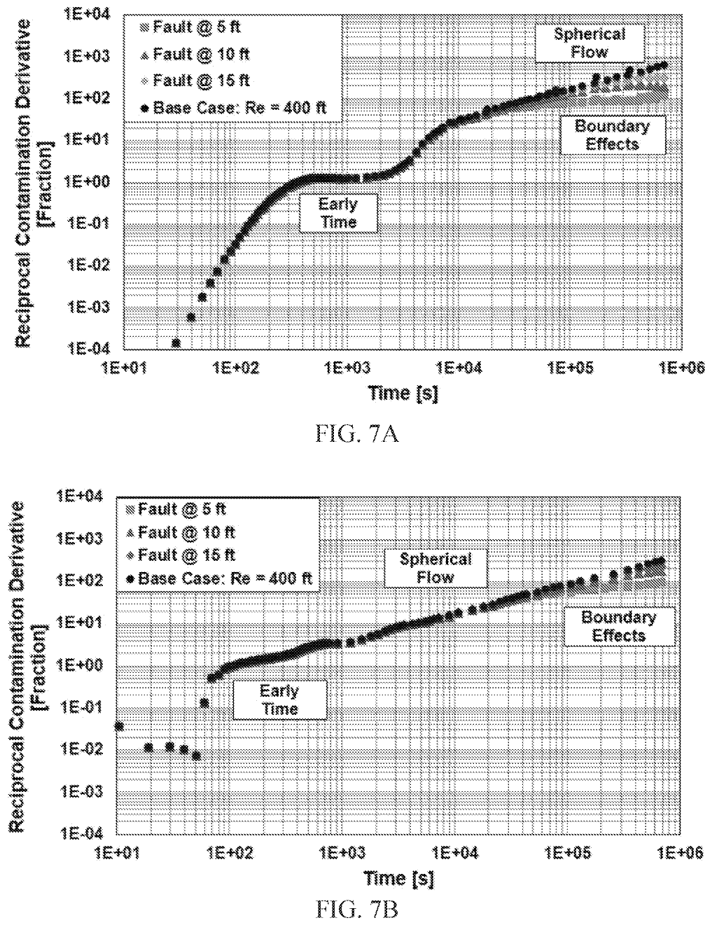

The subsequent simulation cases reproduce the presence of geological faults with radial no-flow boundaries located 5 ft, 10 ft and 15 ft away from the wellbore, where the formation-testing tool is pumping out formation fluids. FIGS. 7A and 7B display the results for this case; again, the early-time regime is the same for all cases. At late-times, however, the ⅔-slope decreases to a lower slope with almost a constant trend. Similarly, this particular horizontal trend occurs first in the model with the fault located closest to the wellbore. The transient response converges to the spherical flow trend as the no-flow barrier is moved radially away from the tool location. These trends are due to the no-flow radial boundaries included in the model.

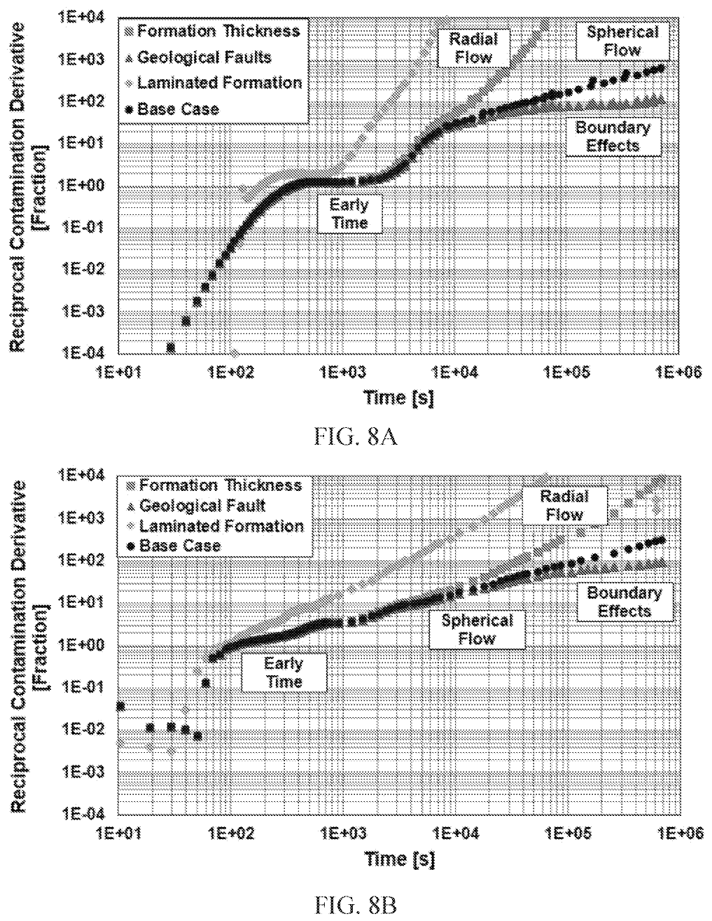

FIGS. 8A and 8B summarize the simulations results in terms of reservoir limits and geometry, confirming the reliability and efficacy of the RCD method to identify spherical flow regimes, radial flow regimes, and boundary effects with the analysis of the transient behavior of contamination measurements during filtrate cleanup.

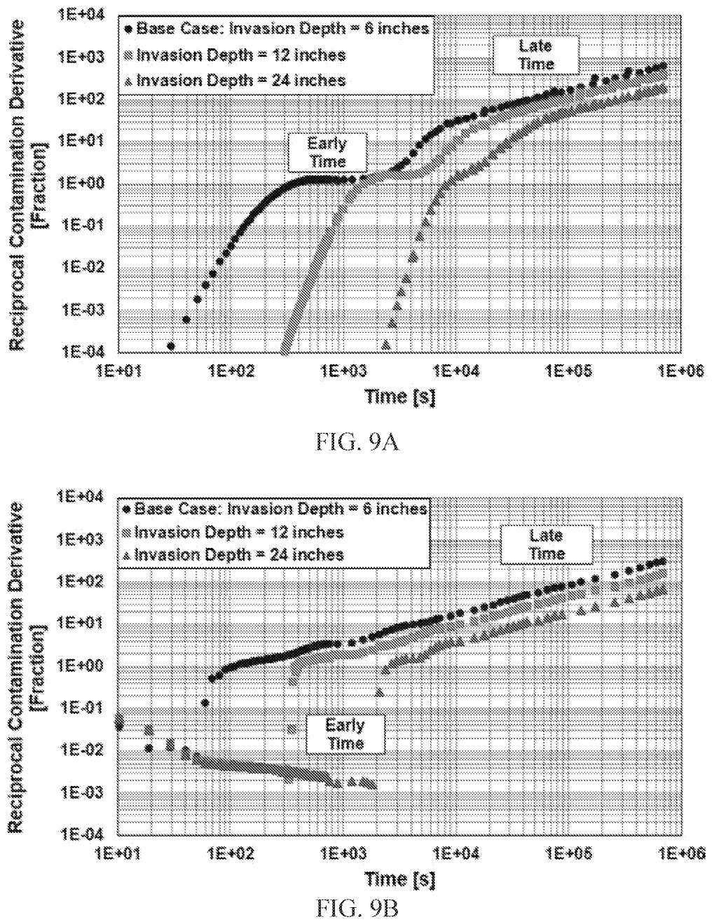

Identification of Reservoir Features. The remaining three simulation cases focus on the behavior of the contamination transient at early times. FIGS. 9A and 9B present the simulation results obtained for the case of mud-filtrate invasion depth. Despite the differences in invasion volume, the three curves exhibit the same trend at late times, confirming that the contamination transient follows the spherical flow regime. On the other hand, at early times the differences are considerable. Short invasion depths give rise to longer horizontal straight lines than the cases of deep invasion. Indeed, this particular early-time regime trend is completely hidden by mud-filtrate invasion for the case of 24-inches of radial length of invasion, and the RCD curve simply exhibits the late-time regime. Therefore, radial length of mud-filtrate invasion plays a role similar to wellbore storage in PTA, which masks the response of the early-time regime in the RCD curve.

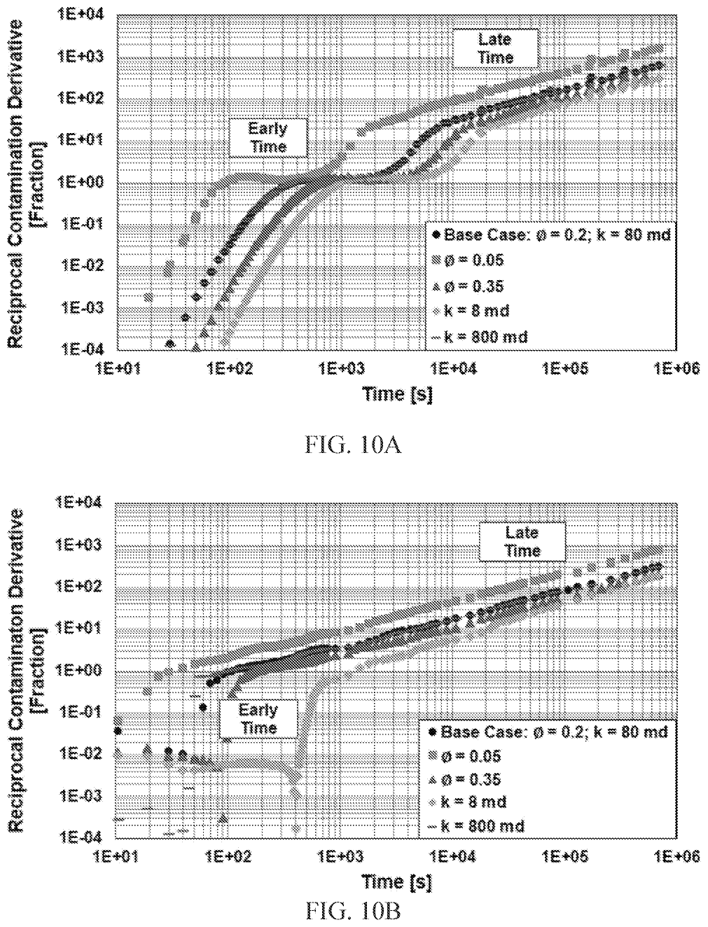

Reservoir properties, such as porosity, permeability, and anisotropy considerably affect the pressure derivative results in pressure transient analysis. Thus, the next simulation cases consider changes of these properties to evaluate their impact and relationship to the contamination transient response. The reservoir properties case investigates the impact of porosity and permeability in the RCD method in which porosities from 5% to 35%, and permeabilities of 8 md to 800 md enable the comparison of these results with respect to the base case (porosity of 20% and permeability of 80 md). FIGS. 10A and 10B show the results of the RCD in terms of these petrophysical properties. All five curves exhibit the same trend for early- and late-time regimes. The only difference is the expected shift in the time domain for both porosity curves due to faster cleanup time as reservoir porosity decreases. Additionally, the high-permeability curve completely overlaps with the base case curve, whereas the low-permeability curve is shifted to the right of the corresponding curve for the base case. This confirms that under equal pump-rate conditions the cleanup time varies but the contamination transient remains constant.

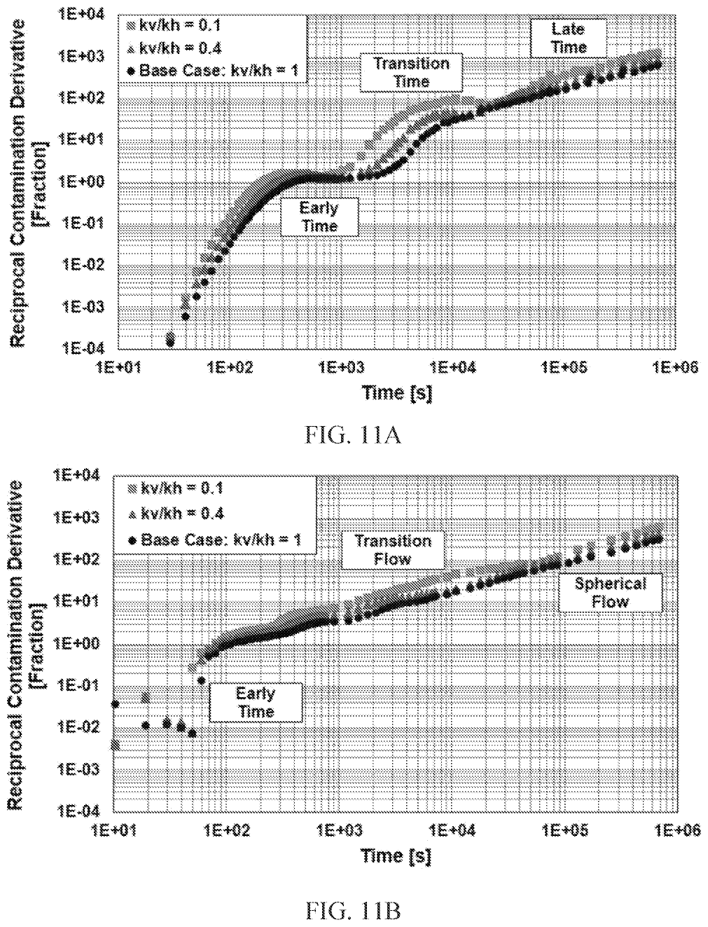

Finally, the permeability anisotropy case studies the impact of a variable permeability as a function of the direction on the RCD method. Multiple anisotropy conditions ranging from an isotropic reservoir to a highly anisotropic formation with a kv/kh ratio of 0.1 are considered to perform the assessment. FIGS. 11A and 11B compares the isotropic case (base case) to kv/kh ratios of 0.4 and 0.1. Early- and late-time regimes tend to converge for the different curves. However, the transition between these two time regimes is not constant: it changes with an increase of anisotropy, with the effect more noticeable for single-phase flow than for multiphase flow.

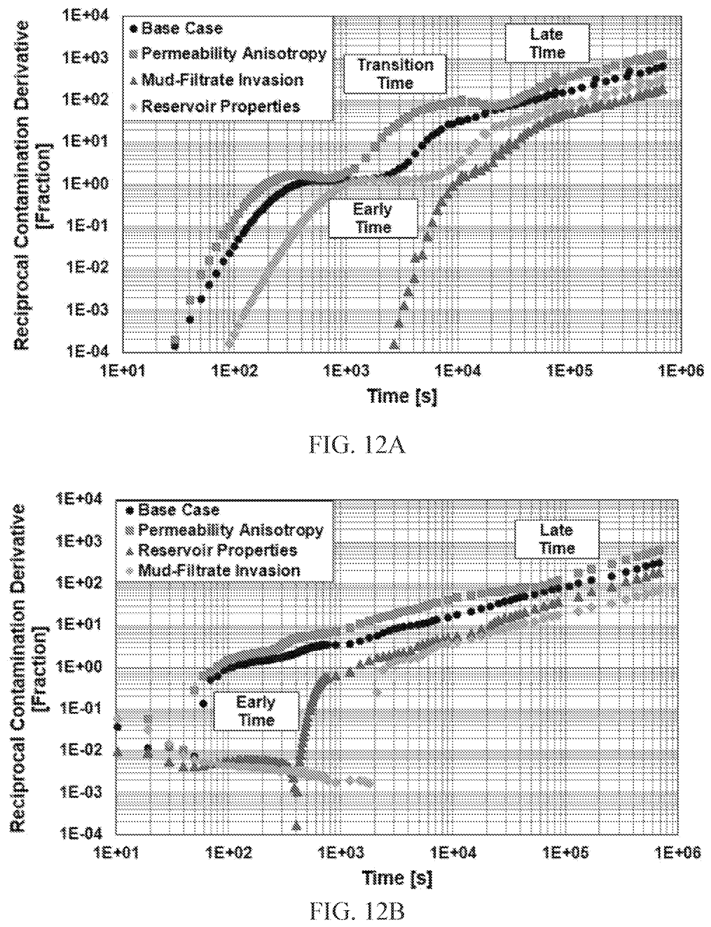

FIGS. 12A and 12B describes the effects of reservoir features in the RCD method. Notably, (1) depth of mud-filtrate invasion affects the early-time regime, (2) permeability anisotropy impacts the transition-time, and (3) the late-time regime is unaffected by either depth of mud-filtrate invasion or permeability anisotropy. Moreover, porosity and permeability do not influence the interpretation of the contamination transient analysis via the RCD method.

LWD Sampling Cases. The first LWD sampling case (House et al., 2015) considers density measurements acquired during cleanup and sampling of an oil-saturated reservoir invaded with oil-base mud-filtrate (single-phase flow). House et al. (2015) studied the application of LWD formation-testing tool sampling in an oil reservoir under active invasion conditions. The following equation is used to calculate the time evolution of fluid contamination based on fluid density measurements:

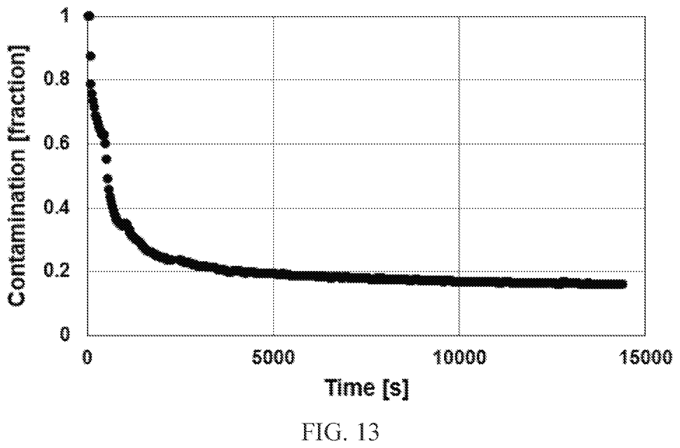

C=(ρ_{measured}−ρᵣₑₛₑᵣᵥₒᵢᵣ)/(ρ_{filtrate}−ρᵣₑₛₑᵣᵥₒᵢᵣ) (2)

where C is contamination, ρ_{measured} is measured density, ρᵣₑₛₑᵣᵥₒᵢᵣ is the actual reservoir fluid density at reservoir conditions, and ρ_{filtrate} is the pure filtrate density at reservoir conditions. FIG. 13 displays the estimated contamination curve, with a final contamination of approximately 16%.

The second case investigates the effects of interruptions while sampling. Because of multiple field conditions, the cleanup operations could be limited in effectiveness; therefore, contamination transient analysis provides an alternative method to interpret and optimize LWD fluid sampling jobs. Density and speed of sound measurements obtained during an LWD operation have been described. These measurements were acquired at the same depth but during two separate runs due to multiple interruptions required to avoid differential sticking of the LWD string. Consequently, both data sets reproduce the change in contamination during fluid cleanup using Equation 2 for density measurements and Equation 3 for speed of sound data, i.e.,

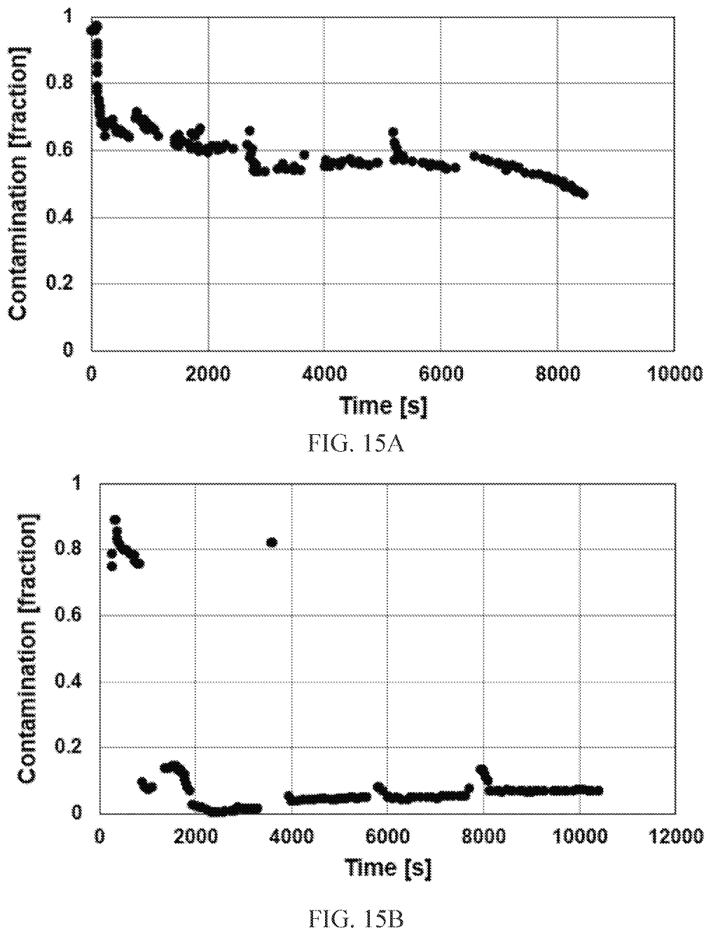

C=(SS_{measured}−SSᵣₑₛₑᵣᵥₒᵢᵣ)/(SS_{filtrate}−SSᵣₑₛₑᵣᵥₒᵢᵣ) (3)

where SS_{measured} is measured speed of sound, SSᵣₑₛₑᵣᵥₒᵢᵣ is the speed of sound in the reservoir fluid at reservoir conditions, and SS_{filtrate} is the speed of sound in pure filtrate at reservoir conditions. As an illustration, FIGS. 15A and 15B describe contamination curves for the first and second run, respectively. Likewise, noise filters are necessary for the subsequent implementation of the reciprocal contamination derivative via a center-point derivative method.

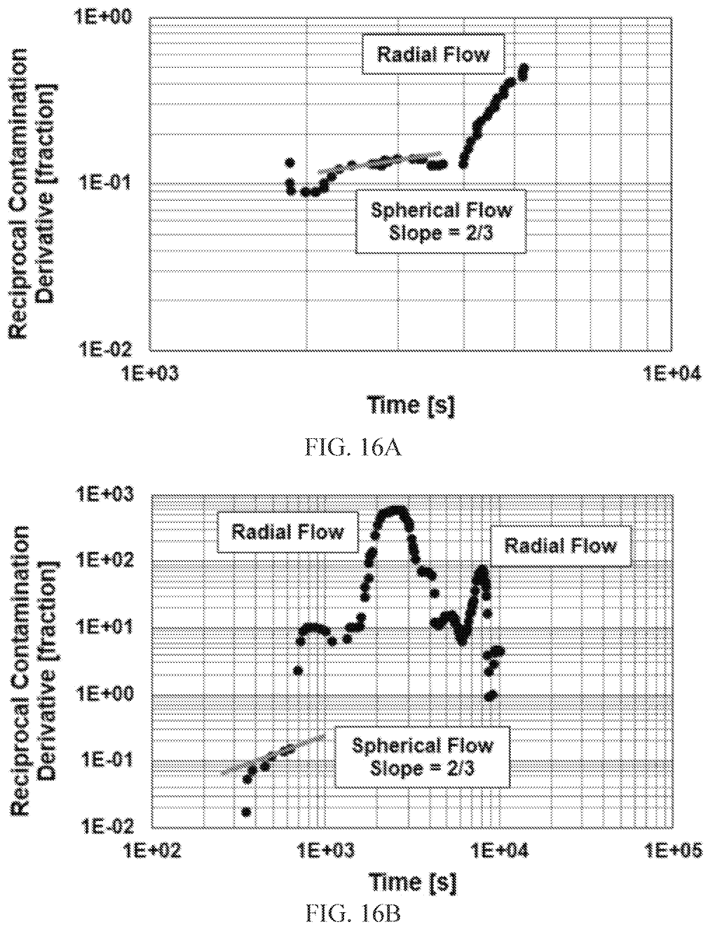

FIGS. 16A and 16B present the RCD results for both runs considered in the second LWD case. The RCD curves reveal the early-time effects of multiphase flow as well as the transition to a late-time regime, where the slope is higher than spherical flow, signaling a radial flow regime. These results confirm the properties of a layered reservoir associated with a turbidite sedimentary system.

Discussion. The RCD method and the CTA concept are an alternative to quantify important reservoir properties. Deviations from late-time trend of the contamination prediction models have been investigated and documented. These deviations have been attributed to the type of probe, whereas the techniques described in this example demonstrate that these trends depend on contamination transient behavior. Late-time regimes include three distinct trends to identify reservoir limits: spherical flow regime (slope equal to ⅔), radial flow regime (greater slope), and boundary effects (constant horizontal straight line). These well-defined late-time regimes provide real-time identification of reservoir layers, thin laminations, and geological faults. Based on numerical simulation results, the RCD estimated length of investigation is approximately 50-ft in the vertical direction and 20-ft in the radial direction. Also, the length of investigation of this technique is strongly related to invasion volume and pumpout time. The RCD method significantly enhances the interpreter's ability to identify the transient behavior of contamination measurements in comparison to conventional fluid cleanup curves.

Qualitative identification of radial length of invasion and movable fluids are additional advantages of the RCD technique. These key reservoir features can be analyzed during early-time regimes. For instance, at early times, the presence of single-phase flow causes a different effect on the RCD curve from that of multiphase flow. Radial length of invasion can also be estimated under single-phase flow conditions, compared to alternative procedures based on well logs which have considerable restrictions and fail to identify the invaded zone. Notably, porosity, permeability, and anisotropy do not significantly affect the RCD interpretation, thus simplifying the evaluation of the corresponding measurements.

In practical applications, the RCD method allows fluid sampling optimization. As confirmed by the results, the RCD analysis facilitates the identification of active invasion during LWD operations and suggests solutions for faster achievement of the contamination target during fluid sampling. Moreover, this transient technique provides additional degrees of freedom for improved interpretation of contamination measurements, which facilitates the detection and quantification of diffusion mechanisms occurring in the near wellbore during mud-filtrate cleanup.

Conclusion. The reciprocal contamination derivative method enables the implementation of contamination transient analysis techniques to investigate late-time flow regimes. It also helps to identify reservoir limits located longer than 20-ft away from the formation-testing tool. These attributes serve to detect and quantify vertical seals, geological faults, and laminated formations by identifying the distinct trends for spherical flow regime, radial flow regime, and no-flow boundary effects. Furthermore, early-time regimes enable the estimation of reservoir fluid types and radial extent of mud-filtrate invasion. The early-time regime is observable with radial lengths of invasion shorter than 2 ft, which provides a qualitative estimation of invasion depth for reliable pump-out decisions. Furthermore, all the benefits of the RCD method are attainable independently of the underlying petrophysical properties, such as porosity, permeability, or anisotropy.

In contrast to PTA, contamination transient analysis is suitable for formation testing applications, especially for LWD fluid sampling, because CTA comprises considerably more data than PTA due to hours of fluid cleanup compared to a few minutes of pressure pre-test. The interpretation technique also avoids the extended buildup period of PTA necessary to reach reservoir limits, thereby saving time and operational costs. Such advantages along with reservoir fluid identification, estimation of radial length of invasion, and detection of reservoir boundaries emphasize the benefits of the RCD method as an innovative formation evaluation procedure. The implementation of this new interpretation approach in real-time LWD operations optimizes sampling time and sample quality acquisition by identifying specific reservoir transient conditions difficult to estimate with contamination measurements and computation of the fluid cleanup curve.

Nomenclature for Example 1

| | | |
|---|---|---|
| C | contamination | fraction |
| 1/C | reciprocal contamination | fraction |
| Δ(1/C) | reciprocal contamination change | fraction |
| h | formation thickness | ft |
| k | permeability | md |
| kv/kh | vertical to horizontal permeability ratio | fraction |
| q | flow rate | cm³/s |
| Re | radial reservoir extension | ft |
| SS | speed of sound | m/s |
| t | time | seconds |
| Δt | elapsed time | seconds |
| ρ | density | g/cm³ |
| Ø | porosity | fraction |
| Subscripts | | |
| i | center point | |
| 1 | location before the center point | |
| 2 | location after the center point | |
| max | maximum | |
| measured | measured property | |
| min | minimum | |

Figure captions for Example 1. FIG. 3A and FIG. 3B: Base case contamination simulation results for (FIG. 3A) single-phase flow, and (FIG. 3B) multiphase flow.

FIG. 4A and FIG. 4B: Base case RCD simulation results for (FIG. 4A) single-phase flow, and (FIG. 4B) multiphase flow.

FIG. 5A and FIG. 5B: Formation thickness case: numerical simulation results for (FIG. 5A) single-phase flow, and (FIG. 5B) multiphase flow.

FIG. 6A and FIG. 6B: Thinly-laminations case: numerical simulation results for (FIG. 6A) single-phase flow, and (FIG. 6B) multiphase flow.

FIG. 7A and FIG. 7B: Geological faults case: numerical simulation results for (FIG. 7A) single-phase flow, and (FIG. 7B) multiphase flow.

FIG. 8A and FIG. 8B: Summary of reservoir boundaries identification cases: results for (FIG. 8A) single-phase flow, and (FIG. 8B) multiphase flow.

FIG. 9A and FIG. 9B: Mud-filtrate invasion depth case: numerical simulation results for (FIG. 9A) single-phase flow, and (FIG. 9B) multiphase flow.

FIG. 10A and FIG. 10B: Reservoir properties case: numerical simulation results for (FIG. 10A) single-phase flow, and (FIG. 10B) multiphase flow.

FIGS. 11A and 11B: Permeability anisotropy case: numerical simulation results for (FIG. 11A) single-phase flow, and (FIG. 11B) multiphase flow.

FIGS. 12A and 12B: Summary of reservoir features identification cases: results for (FIG. 12A) single-phase flow, and (FIG. 12B) multiphase flow.

FIG. 13: Cleanup curve for LWD fluid sampling case 1.

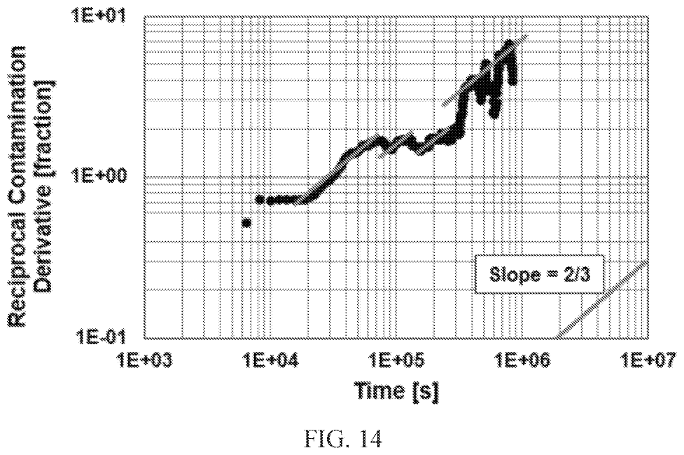

FIG. 14: RCD method for LWD fluid sampling case 1.

FIG. 15A and FIG. 15B: Cleanup curves for LWD fluid sampling case 2: (FIG. 15A) Run 1, and (FIG. 15B) Run 2.

FIG. 16A and FIG. 16B: RCD method for LWD fluid sampling case 2: (FIG. 16A) Run 1, and (FIG. 16B) Run 2.

### Example 2: Fluid Contamination Transient Analysis

Successful in situ fluid cleanup and sampling operations are commonly driven by a fast and reliable analysis of pressure, rate, and fluid contamination measurements. Techniques such as pressure transient analysis (PTA) provide important information to quantify reservoir complexity, while fluid contamination measurements are commonly overlooked for reservoir characterization purposes. This Example introduces a new interpretation technique to relate fluid contamination measurements with near-wellbore parameters by identifying early- and late-time flow regimes in fluid contamination and its derivative function.

The derivative methods used in PTA inspired the development of the new fluid contamination interpretation method. Contamination transient analysis (CTA) evaluates transient measurements acquired during cleanup of mud-filtrate invasion to infer important reservoir geological and flow conditions. This Example describes application of center-point derivative methods to the pumpout volume and time evolution of fluid contamination to identify flow regimes in cases of water-base mud invading either water- or hydrocarbon-bearing formations.

This Example documents synthetic examples of the new interpretation method for seven reservoir cases, numerically simulated to obtain contamination data for: homogeneous isotropic reservoir, radial boundaries, vertical boundaries, thin laminated formations, mud-filtrate invasion radius, petrophysical properties, and permeability anisotropy. In addition, single-phase flow and multiphase flow cases are compared.

The new approach of the fluid contamination derivative (FCD) an provide an alternative to optimize fluid cleanup efficiency and to detect the spatial complexity of the reservoir during real-time downhole fluid sampling. Using log-log plots of fluid contamination and the FCD method, characteristic slopes may be encountered, defining late-time flow regimes. Spherical flow regime presents a slope of −⅔, which has been documented by homogeneous isotropic analytical models. Radial flow presents a steeper slope of −3 that can be detected when the vertical limits are attained. Likewise, boundary effects are evident when the late-time slope of the FCD is equal to −⅓. In addition to the detection of reservoir boundaries, the CTA techniques presented in this Example enable the identification of reservoir fluid type and shale laminations, and could potentially provide a foundation for the quantification of invasion radius and permeability anisotropy.

Introduction. Changes in fluid contamination are continuously measured during real-time cleanup operations to assess fluid sample quality. This is currently the sole use for contamination data despite the great amount of information collected over hours of fluid pumpout. Post-job applications often overlook cleanup measurements. In contrast, pre-test pressure data provide formation pressure measurements, vertical connectivity, and fluid contacts, while pressure transient analysis (PTA) estimates reservoir mobility and vertical boundaries in thin layers. However, both short pre-test duration and small volume of investigation significantly limit PTA techniques for formation testing applications. This Example describes a new transient analysis method based on fluid contamination measurements to enhance formation-testing capabilities in reservoir description and potentially improve fluid cleanup efficiency in real-time.

In addition to its remarkable value for reservoir evaluation, PTA methods, such as pressure derivative and pressure convolution serve to develop analogous methods for contamination transient assessment. Pressure derivative methods assist to identifying flow regimes, reservoir geometry, and formation boundaries. Furthermore, such pressure derivative can apply a center-point technique, which considerably reduces the effect of noise in the derivatives computation. Noise reduction can be useful for real-time measurements because the acquired data often exhibit large noise-to-signal ratios.

The novel transient analysis method described herein has the potential to positively impact formation-testing operations when used to characterize reservoir complexity and interpret cleanup trends to achieve optimal fluid sampling. Information about reservoir geometry and boundaries, presence of grain-size laminations, invasion radius, and anisotropy are key to optimize fluid pumpout. This new transient analysis approach expands the proficiency of formation testing and overcomes the limitations of PTA in fluid sampling operations, such as build-up time restrictions. The following sections of this Example introduce the concept of fluid contamination transient analysis and a new derivative approach on fluid cleanup measurements to further examine several numerical simulation cases to demonstrate the value of fluid contamination transient methods in reservoir description and fluid sampling operations.

Fluid Contamination Transient Analysis. Fluid contamination transient analysis (CTA) can be defined as a novel reservoir evaluation technique that studies the transient response of mud-filtrate concentration during downhole fluid cleanup and sampling. Fluid-flow transients are strongly related to flow geometry. Transient trends, observed for various fluid properties, reflect the distribution and structure of the flow. In formation-testing applications, pressure transients serve to define the effect of flow geometry during pressure drawdown and pressure build-up periods. Similarly, fluid contamination transient could potentially identify flow geometry. The effect of flow geometry in filtrate cleanup efficiency has been demonstrated and the fluid contamination response during downhole sampling, for various types of formation testers and probes, has also been shown. Moreover, transient flow regimes provide a description for flow geometry in the near-wellbore in the proximity of the formation-testing tool. It is reasonable to assume that the fluid-flow transient response can be approximated to a spherical flow regime in the vicinity of the probe. In this region, the flow geometry obeys to a spherical reservoir system because the reservoir flow pattern converges toward a point probe. Spherical flow regime will govern the flow geometry until the flow distribution attains a reservoir boundary. If the formation is vertically bounded, the flow pattern switches to a cylindrical flow geometry that enables a radial flow regime. Radial flow regime occurs when the fluid transient observes the vertical boundary, but it does not encounter an outer radial boundary. During this flow regime, the flow direction is perpendicular to the formation tester axis.

Furthermore, flow geometry changes in the near-wellbore region during downhole fluid cleanup and sampling, which permits the identification of flow regimes throughout the evaluation of fluid contamination measurements. Fluid contamination is an estimated parameter which assess the mud-filtrate fraction in the fluid sample as a function of pumpout volume and time. According to the mixing rules for various fluid properties, the fluid contamination (C) is defined, in Eq. 4, as a normalized estimation of any downhole fluid property measurement with respect to its virgin reservoir fluid and mud-filtrate known-values.

$$C = \frac{{φ}_{( V , t )} - {φ}_{f}}{{φ}_{mf} - {φ}_{f}} , \qquad ( 4 )$$

where C is fluid contamination, φ stands for any fluid property measured with a formation tester; V is pumpout volume and t is cleanup time; the subscripts f and mf represent the reservoir fluid and mud-filtrate, respectively.

Fluid contamination can also be expressed mathematically as a power-law function of time or pumped volume. This Example investigates the effect of transient behavior and flow regimes in these types of power-law functions and models. Another analytical model was used to describe and predict mud-filtrate cleanup performance, which assumes a point probe and a spherical flow approach to define the fluid contamination as a function of time and pumpout volume. A mathematical expression is used in this model that approximates the fluid contamination with time to the power of −⅔ (t^{-2/3}). Simulations have previously been performed to generate synthetic cases and evaluate the effect of various parameters in fluid cleanup time, such as boundary effects, invasion radius, pumpout rate, porosity, permeability anisotropy, fluid viscosity ratio, fluid density difference, capillary pressure, relative permeabilities and end-point mobilities. An analytical model can be used to predict fluid contamination with a non-constant time power value that depends on the invasion radius and the end-point mobility ratio. No-flow boundaries were observed to reduce cleanup time and an empirical correction factor can be used to adapt the time scale during filtrate cleanup, taking into account the effects of reservoir boundaries in the time evolution of the fluid contamination. Optical spectroscopy data obtained with downhole formation testers can be matched using a power t^{-5/12}. Fluid cleanup simulations for diverse formation testing tools and reservoir conditions have been performed previously, identifying three different flow regimes: short period of pure filtrate pumpout, intermediate period, and late-time period. These three flow regimes have been observed in fluid cleanup simulations for diverse formation-testing tools and reservoir conditions. In general, these techniques define the late-time period as a developed flow regime proportional to t^{-2/3}. This Example classifies these flow regimes as early- and late-time flow regimes, and describes a derivative method that contributes to the identification of flow regimes and provides new applications for fluid contamination measurements.

Fluid Contamination Derivative Method. This Example describes a derivative approach for the fluid contamination transient, which enables the definition of late-time flow regimes and identifies factors affecting early-time flow. The fluid contamination derivative (FCD) method is implemented using a center-point derivative method. The corresponding numerical implementation of the FCD method obtained is expressed in Eq. 5:

$${( \frac{dC}{d ln V} )}_{i} = {( V \frac{dC}{dV} )}_{i} = \frac{\frac{{C}_{i} - {C}_{L}}{ln ( {V}_{i} / {V}_{L} )} ln ( {V}_{R} / {V}_{i} ) + \frac{{C}_{R} - {C}_{i}}{ln ( {V}_{R} / {V}_{i} )} ln ( {V}_{i} / {V}_{L} )}{ln ( {V}_{R} / {V}_{L} )} \qquad ( 5 )$$

where V is pumpout volume, C is fluid contamination; subscripts i, L (left) and R (right) designate the center-point data and its data before and after, respectively.

Eq. 5 describes the fluid contamination derivative with respect to the pumpout volume (V). For the calculations made in this Example, fluid contamination is defined as a function of pumpout volume because it can eliminate the relative effects of flowrate and fluid cleanup time inefficiencies. However, this derivative approach can also be implemented as a function of time (t) by replacing the pumpout volume data for the time data to obtain the following expression in Eq. 6:

$${( \frac{dC}{d ln t} )}_{i} = {( t \frac{dC}{dt} )}_{i} = \frac{\frac{{C}_{i} - {C}_{L}}{ln ( {t}_{i} / {t}_{L} )} ln ( {t}_{R} / {t}_{i} ) + \frac{{C}_{R} - {C}_{i}}{ln ( {t}_{R} / {t}_{i} )} ln ( {t}_{i} / {t}_{L} )}{ln ( {t}_{R} / {t}_{L} )} . \qquad ( 6 )$$

Moreover, downhole fluid sampling data is commonly acquired at a high sampling rate, which increases the noise on the derivative response. Presence of noise can make the evaluation of the FCD curve and flow regimes more difficult. A scattered response in the FCD can be observed if fluid contamination sampling rate or noise-to-signal ratio are high, and data points L and R consecutive to i can be selected. Likewise, if the distance between these differentiation data points is high, the FCD response can be altered. To overcome noise or over smoothing difficulties, the center-point derivative technique employs a minimum distance between the extreme data points (L or R) and the differentiation point (i). This differentiation interval is referred to as the smoothing factor (X). This smoothing factor can be applied with an adjustable window in the log scale and with a maximum value of ln(V_{R}/V_{L}). In addition, end effects can be considered when the ith point is close to the first or last data point. For these edge periods, V_{R} and V_{L} can be fixed with the last and first data point within the fluid contamination dataset, respectively. The proper assessment of the smoothing factor is useful for the success of the FCD technique. Therefore, an adjustable X varying from 0 to ln(V_{R}/V_{L}) can be used, depending on the noise in the fluid contamination signal.

However, the FCD may still exhibit a noisy response after employing the maximum smoothing factor. Noise filters may be applied to enable a reliable and accurate assessment of fluid contamination measurements and subsequent calculation of the contamination transients via the FCD method. Presence of noise in the measurements can be a major concern in derivative approaches, where the calculation of the derivative implicitly enhances the effect of noise. To overcome this challenge, a noise filter can be implemented on the fluid measurements acquired by the formation tester. This filter can calculate the median of the data using an independent and adaptable window, which automatically adjust its length on a logarithmic scale according to the noise-to-signal-ratio and the stages of fluid cleanup and sampling. For the downhole fluid measurements dataset, the noise filter can replace each data entry with the median value of the neighboring data included in the adjustable window. Therefore, the median filter is suitable for the interpretation of fluid contamination data because it eliminates the impact of data outliers commonly encountered during pumpout operations. The noise median filter assists the center-point method to accurately compute the FCD curve for a proper visualization of the fluid contamination transient and its flow regimes.

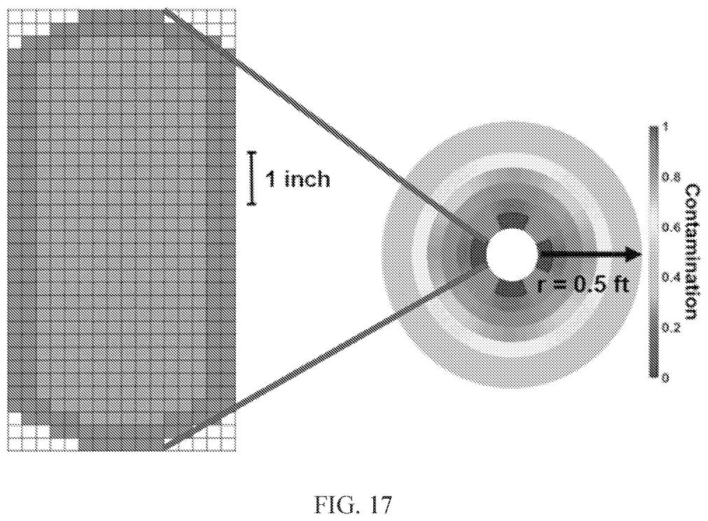

Numerical Simulations. Numerical simulation is a useful step to validate the FCD technique under diverse reservoir conditions. To that end, a compositional numerical model is used to reproduce water-base mud (WBM) filtrate invasion and fluid sampling in a water- (single-phase flow) and a hydrocarbon-saturated formation (multiphase flow). Both models employ a cylindrical grid refined in the near-probe and near-wellbore regions to accurately simulate the complexity of fluid flow phenomena taking place in the invaded zone. All the numerical simulation cases described below assume a radial probe, which uses four probes equally-spaced in the axial direction around the tool and in contact with the wellbore. This multiprobe arrangement permits the reservoir fluid to flow from all directions towards the probe, which favors the development of a spherical flow geometry in the near-wellbore region at larger fluid volumes than those observed for a single-point-probe. FIG. 17 illustrates a top view of the simulation-model near-wellbore region and the refined grid implemented on each of the four probes of the radial tool. After generating and initializing the numerical model, a history matching of fluid cleanup measurements can be performed to calibrate the near-wellbore and formation-testing synthetic system. The construction and validation of numerical models provide quality control for the output data required for testing the contamination transient technique.

The numerical simulations can be performed using commercially available software. The novel transient analysis technique can be evaluated for single-phase flow and multiphase flow using synthetic data obtained from a compositional model and a black oil model, respectively. The compositional model reproduces a blue-dye tracer WBM invading a water-saturated formation. A blue-dye tracer component can be used in the mud-filtrate phase to differentiate the mud-filtrate from the formation water. For the compositional model, mud-filtrate and in-situ reservoir fluids are fully miscible. Similarly, a black oil model can be employed for reproducing a multiphase flow case with a WBM invading a hydrocarbon-saturated reservoir. Once the models are verified and benchmarked for their accuracy and reliability, seven reservoir simulation cases are constructed to obtain synthetic data to implement the FCD method: Base case: homogeneous isotropic reservoir, radial boundaries, vertical boundaries, thin laminations, mud-filtrate invasion, reservoir properties, and permeability anisotropy.

The sensitivity analysis for single-phase flow and multiphase flow numerical simulation models provide similar information. Therefore, the results for all simulation scenarios are described for the single-phase flow models. The transient and FCD response are compered for single-phase flow and multiphase flow only for the base case. In addition, the effect of the smoothing factor (X) on the FCD signature is investigated, and an adequate range is defined to reduce the noise in the derivative computation and at the same time avoid oversmoothing.

Base Case: Homogeneous Isotropic Reservoir. The base case uses a clean homogeneous-isotropic infinite reservoir model, which provides a suitable reference for comparison of various reservoir conditions throughout numerical simulations and sensitivity analyses. Table 2 presents the input parameters of the base case simulation model.

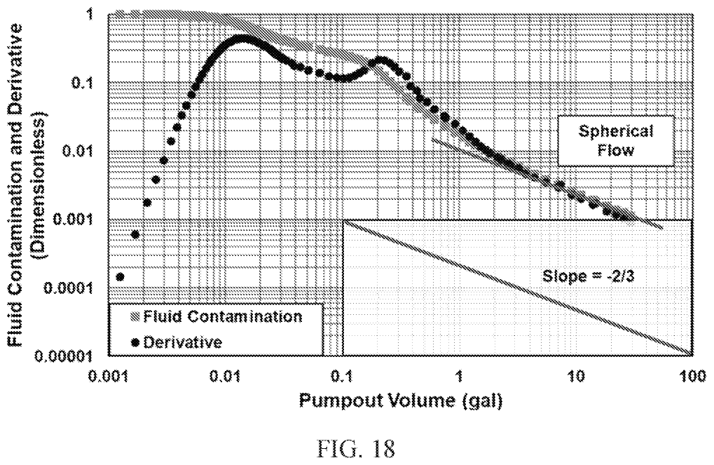

The fluid contamination data obtained from the simulation results of the base case model is used to generate a reference signature trend for both, fluid contamination and the FCD. FIG. 18 shows the fluid contamination and FCD curves for the base case model. The log-log plot includes notable features, such as the V^{-2/3} late-time trend in the contamination curve. The FCD exhibits distinct trends for early- and late-time regimes. The early-time regime presents a double hump, while the late-time regime displays a slope coinciding with the V^{-2/3} trend of the fluid contamination curve. This late-time regime, identified with a slope equal to −⅔ in a log-log scale, coincides with a spherical flow model. In addition, the infinite-boundary base case model ensures the presence of a spherical flow regime at late times.

TABLE 2
Numerical simulation model input parameters for the base case.

| Parameter | Value | Units |
|---|---|---|
| Reservoir thickness (h) | 100 | ft |
| External radius (Re) | 400 | ft |
| Wellbore radius | 0.354 | ft |
| Total porosity (Ø) | 0.20 | fraction |
| Permeability (k) | 80 | md |
| Reservoir pressure | 1890.75 | psia |
| Permeability Anisotropy (kv/kh) | 1.0 | fraction |
| Mud-filtrate viscosity | 1.0 | cp |
| Formation fluid viscosity, single-phase flow | 1.0 | cp |
| Formation fluid viscosity, multiphase flow | 2.0 | cp |
| Invasion radius | 6.0 | in |
| Invasion time | 12 | hours |
| Maximum pumpout rate | 27 | cm³/s |
| Maximum drawdown pressure | 190 | psia |

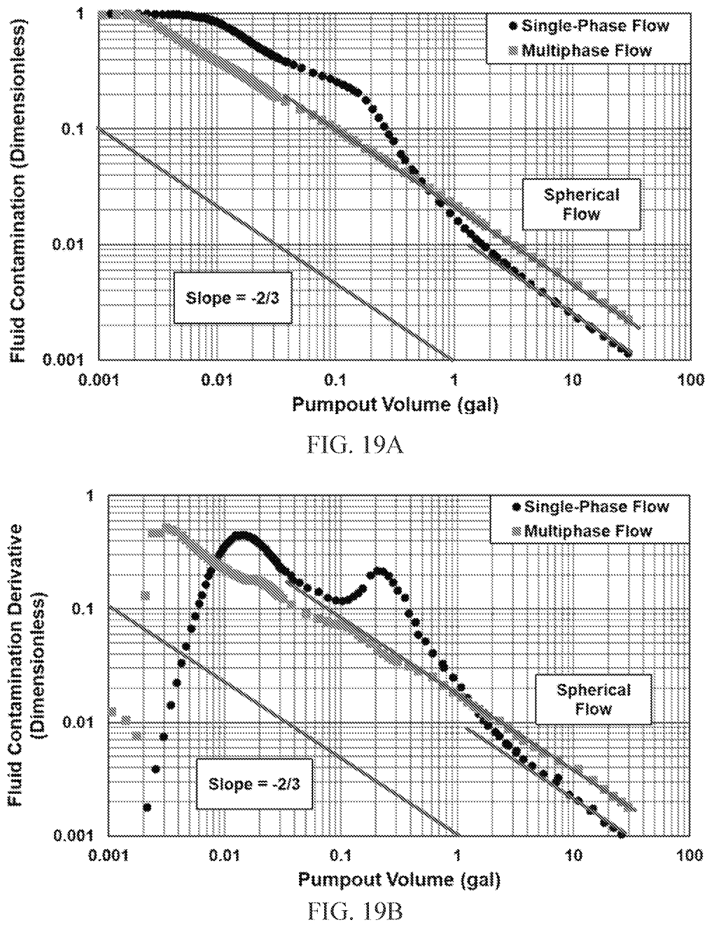

The multiphase flow results are compared in FIG. 19A and FIG. 19B. The late-time spherical flow regime is observed earlier in the multiphase flow case than in the single-phase flow results. Since both models have exact WBM mud-filtrate properties and general numerical simulation conditions, the only difference is the type of fluid saturating the reservoir (water for the single-phase flow and oil for the multiphase flow model). This suggests that early-time flow regimes are heavily influenced by the invaded zone interaction between mud-filtrate and reservoir fluid. Miscibility and dispersion within the near-wellbore region could be the factors generating these different responses. Fluid contamination estimation in single-phase flow scenarios is highlighted as challenging because of the similar values of mud-filtrate and reservoir fluid properties. Indeed, the combination of the fluid contamination and FCD curves might not only enable the identification of reservoir fluid type, but also help reduce the uncertainty in the contamination estimation during cleanup.

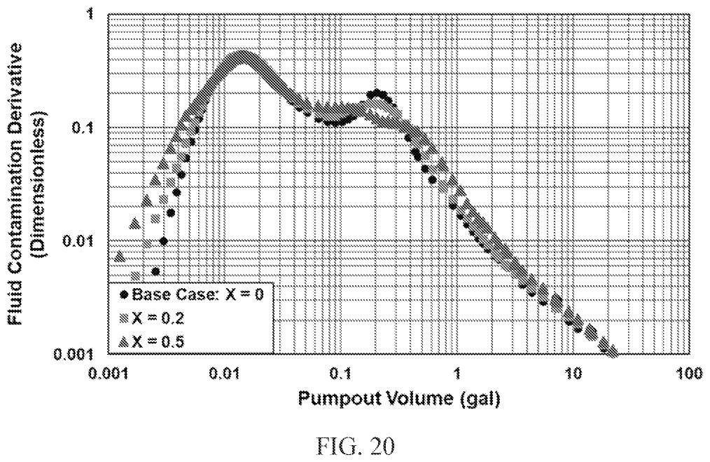

In order to validate the derivative method and its signature, a sensitivity analysis was developed for the smoothing factor, X In FIG. 20, the late-time trend appears less affected than the early-time trend, with increasing X A maximum differentiation interval of 0.5 may be used. At this value, the adjustable window provides good results, and the FCD distortion is not relevant to render wrong interpretation of flow regimes. However, the length of the smoothing factor would depend on the type of application and the quality of the downhole fluid data. Values of X below of 0.5 may be advantageously used to avoid over smoothing. In some cases, a noise median filter can optionally be used on the fluid contamination measurements, before computing the FCD.

Identification of Reservoir Boundaries. No-flow barriers are placed at different vertical and radial distances from the modeled formation-testing tool, respectively. These cases are extremely useful to estimate reservoir limits via transient analysis, enabling ideal conditions for the investigation of flow regimes. In addition, thin-layered models are constructed to reproduce the effects of shale laminations on both mud-filtrate invasion and fluid cleanup, and offer a more complex case for the identification of flow regimes using the FCD method.

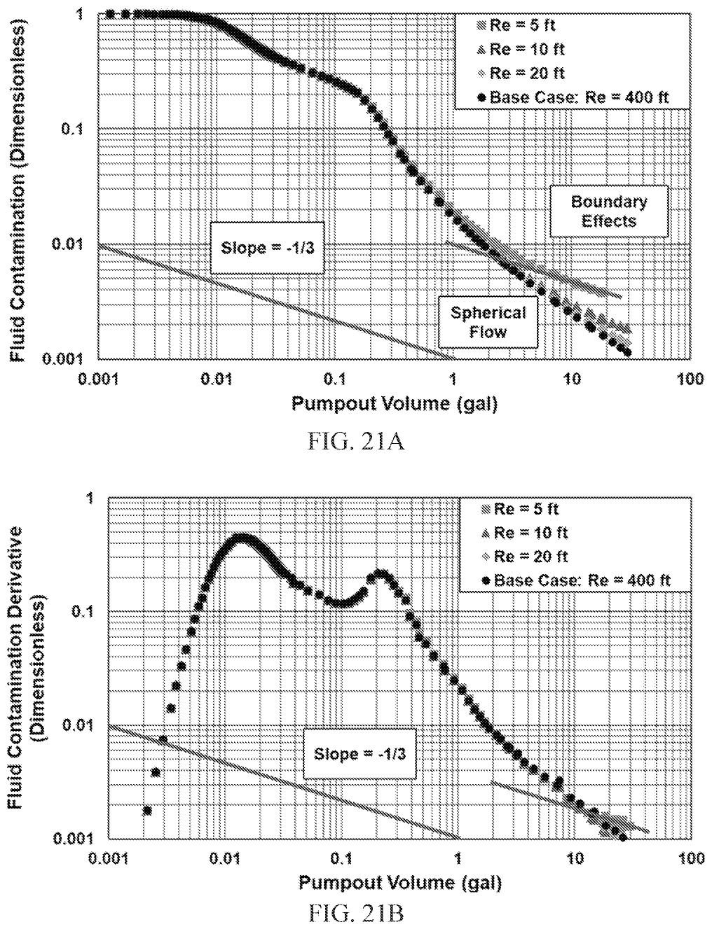

The radial boundaries numerical simulation cases employ radial no-flow boundaries located 5 ft., 10 ft., and 20 ft. away from the wellbore to reproduce the external radius (Re) of finite reservoirs, such as reservoirs bounded by geological faults. FIG. 21A and FIG. 21B display the results for the radial boundaries case. No observable differences exist during the early-time regime. At late-times, however, the fluid contamination trend changes, showing a slope of −⅓ when Re=5 ft. This slope change suggests that the fluid contamination transient reached the radial boundary. This transient behavior is denoted as boundary effects. From FIG. 21A and FIG. 21B, fluid contamination curve is observed to provide a higher resolution than the FCD for the identification of boundary effects. In addition, the transient response converges to the spherical flow slope as the no-flow barrier is moved radially away from the tool location, which indicates that the fluid contamination transient is no longer sensing the effects of the radial boundary. Spherical flow regime occurs before the fluid contamination transient attains a radial boundary located at 20 ft. Similarly, boundary effects can be evaluated at a location of 5 ft. away from the probe.

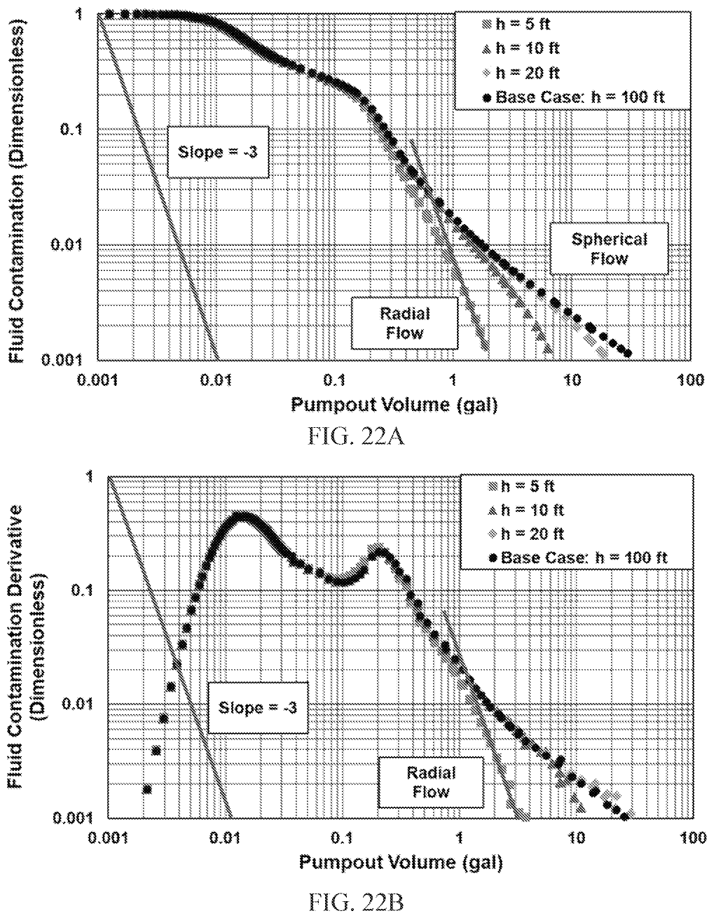

Furthermore, the subsequent reservoir simulation cases consider the presence of vertical limits at several distances from the formation-testing tool to investigate the effect of vertical boundaries in the fluid contamination transient. These cases are reproduced by reducing the formation thickness (h) on the base case model from 100 ft. to 20 ft., 10 ft., and 5 ft., respectively. FIG. 22A and FIG. 22B show the fluid contamination and FCD plots and sensitivity analysis for these numerical simulation cases. Again, the early-time regime is the same for all cases; by contrast, notable differences arise during the late-time regime. All fluid contamination and FCD curves deviate from the spherical flow regime observed in the base case. As formation thickness decreases, the fluid contamination and the FCD late-time trends approach to a slope of −3. This late-time pattern could indicate a change in the flow regimes from spherical flow to radial flow. In transient analysis methods, radial flow regime is attained when the transient response reaches the vertical seals of the formation. In our simulations, we observe a disturbance in the flow geometry of the invaded region. Consequently, our simulation results and log-log plots reveal the effects of vertical limits in the fluid contamination transient, enabling the identification of a radial flow regime.

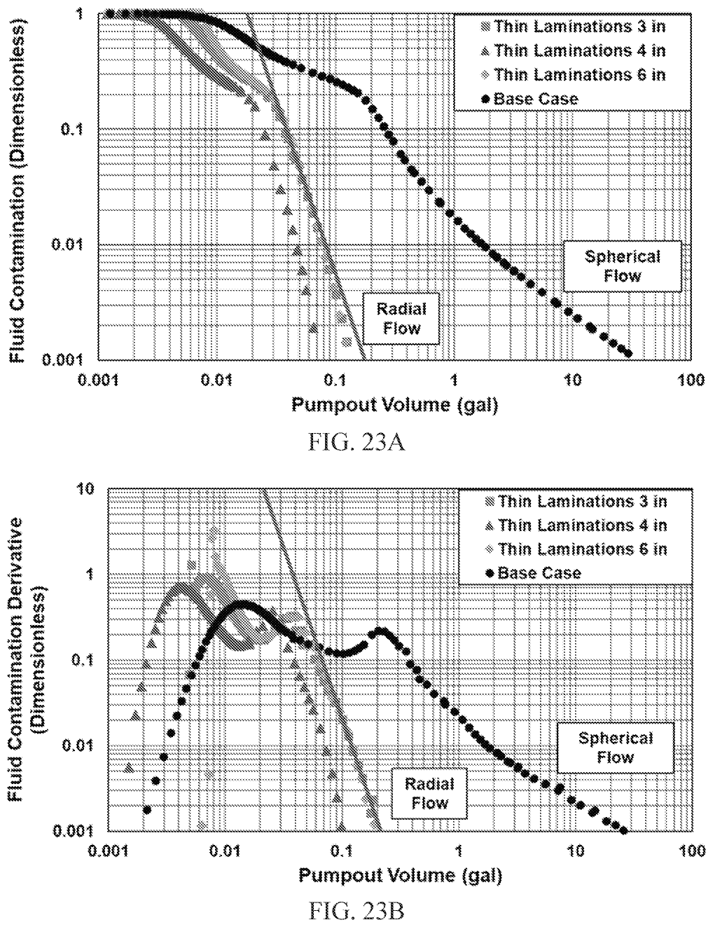

To further validate the observations for the previous case, three different reservoir simulation models were developed with thin-laminations of 3 in., 4 in., and 6 in., respectively. These reservoir models reproduce the effect of complex laminated systems during fluid cleanup and sampling. Likewise, these cases serve to investigate the effects of the proximity of vertical seals since these shale laminations are modeled as no-flow vertical barriers in the numerical simulations. FIG. 23A and FIG. 23B compare the base case with the thin-laminations reservoir models. The differences in the early-time patterns emerge due to differences in the invasion process. Since invasion conditions are equal for all simulation cases, the different bed thickness and their location with respect to the formation tester probe have direct implications on flow geometry in the near-probe region of thin laminated reservoirs. Nevertheless, these effects are not observed in the late-time flow geometry and the fluid contamination transient plots, where all thin-laminations curves show a slope of −3. The late-time regimes exhibit a steeper slope similar to the vertical boundaries case response. Indeed, the late-time slope of −3 confirms the effect of vertical limits on the fluid contamination transient and the presence of the radial flow regime. In addition, the radial flow regime emerges earlier in thin beds because the contamination transient senses the vertical limits faster in these simulations than in the vertical-boundaries cases. Interestingly, the three laminated reservoir curves almost overlap and present the same slope at late-times, allowing easier detection of shale laminations via the fluid contamination and FCD plots.

Identification of Reservoir Features. The remaining three numerical simulation cases focus on the early-time behavior of the fluid contamination transients. These cases intend to identify the signature of the transient response during fluid cleanup and sampling for the assessment of invasion radius, and key petrophysical properties in the near-wellbore region.

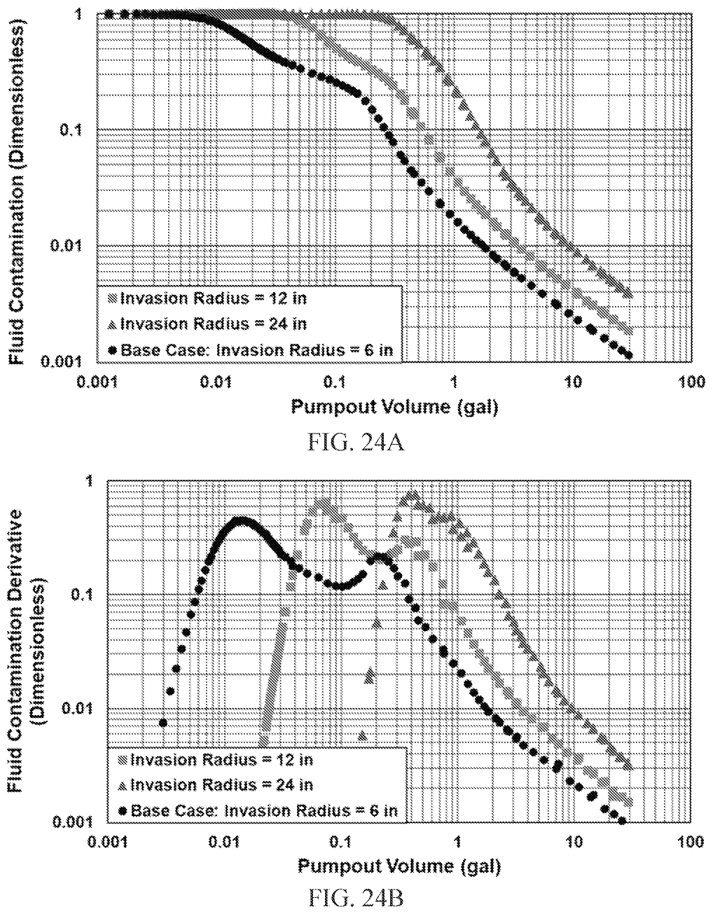

The uncertainty of invasion radius can be assessed during fluid sampling operations varying the invasion time of the base case model to obtain multiple reservoir scenarios with variable invaded regions. Therefore, the process of mud-filtrate invasion is simulated under different drilling conditions to obtain several invasion radii for comparison. For these two additional cases, the invasion time is extended to 24 hours and 48 hours, respectively. With these invasion conditions, two scenarios are reproduced with an invaded region of 12 in. and 24 in., respectively. FIG. 24A and FIG. 24B shows the simulation results obtained for the case of variable mud-filtrate invasion radius. Despite the differences in invasion time and volume, the three curves exhibit the same trend at late-times, confirming that the contamination transient follows the spherical flow regime, once it seizes the near-wellbore effects. On the other hand, at early-times the differences in the FCD curves are considerable. Shorter invasion radius gives rise to wider separation between the two distinct humps of the FCD early-time patterns. The second hump of the early-time regime trend is completely hidden by mud-filtrate invasion for the invasion radius of 24 in., and the FCD curve exhibits only one hump before the late-time slope of −⅔. Apparently, mud-filtrate invasion radius plays a role similar to wellbore storage in PTA, which masks the response of the early-time regime in the FCD curve.

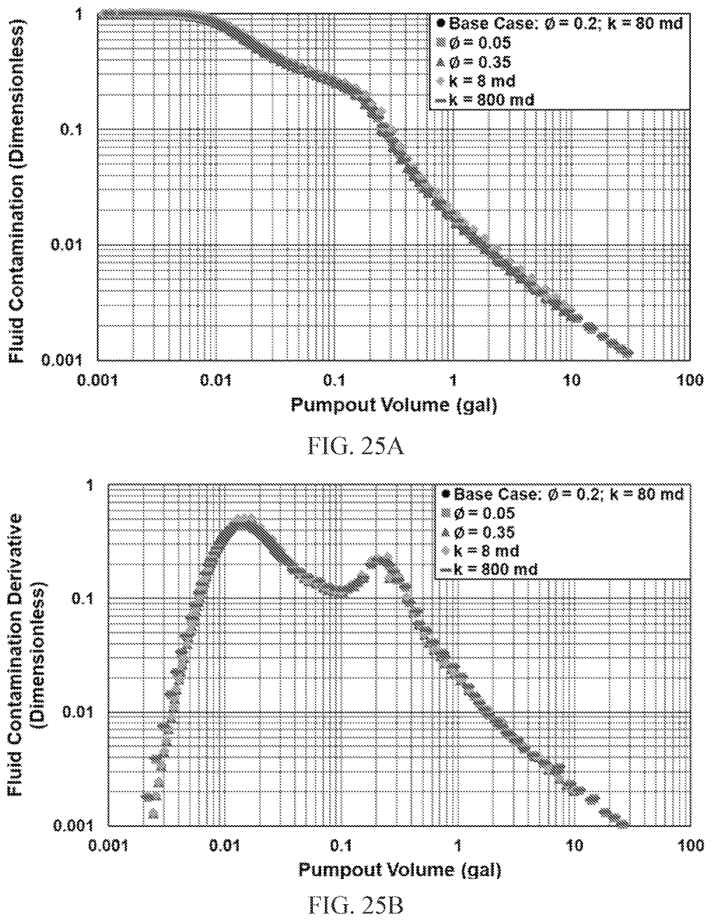

Reservoir properties, such as porosity, permeability, and anisotropy are other parameters that considerably affect the pressure derivative results in pressure transient analysis. Thus, the next simulation cases consider changes of these properties to evaluate their impact on the fluid contamination transient response. The reservoir properties cases investigate the impact of porosity and permeability on the fluid contamination log-log plot and the FCD method. A sensitivity analysis is performed, varying total porosity from 5% to 35%, and reservoir permeabilities from 8 md to 800 md. FIG. 25A and FIG. 25B show the fluid contamination and FCD curves for these petrophysical properties cases. All five curves completely overlap and exhibit the same trend for both plots, the log-log fluid contamination and the FCD. This behavior confirms that under equal pumpout conditions the cleanup time may vary but the fluid contamination transient remains constant. Even though this result implies that fluid contamination transient techniques are not suitable for petrophysical properties calculations, it demonstrates that fluid cleanup efficiency and flow geometry in the near-probe region are not affected by total porosity and formation permeability.

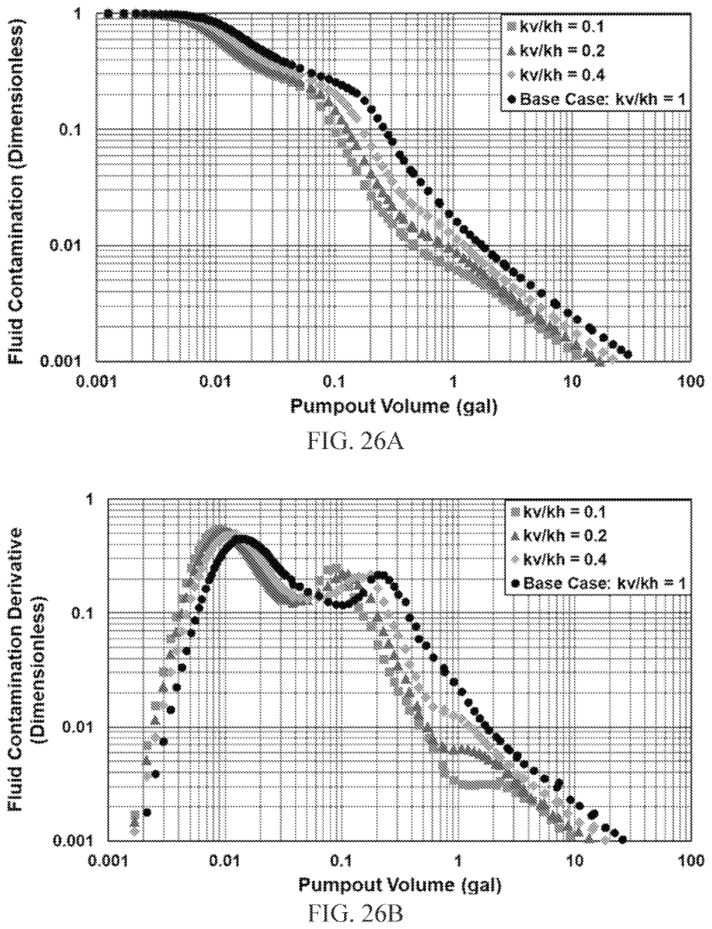

The final numerical simulation case studies the impact of permeability anisotropy on fluid contamination and the FCD method. Multiple anisotropy conditions are considered, ranging from the base case isotropic reservoir model to a highly anisotropic formation with a vertical-to-horizontal-permeability (kv/kh) ratio of 0.1. FIG. 26A and FIG. 26B compares the log-log fluid contamination and the FCD response for the base case isotropic reservoir model (kv/kh=1) with three anisotropic reservoir models with kv/kh ratios of 0.4, 0.2, and 0.1, respectively. For this scenario, the FCD curves show clear differences between 0.2 and 2 gal of pumpout volume. These differences are more evident in the FCD plot than in the log-log fluid contamination. Early- and late-time regimes tend to converge for the different curves, but the transition between these two flow regimes is not constant: it changes with an increase of anisotropy. After the derivative second hump, the FCD pattern presents a slope close to −3 before achieving the spherical flow slope. The greater the anisotropy, the greater the required pumpout volume to observe the spherical flow regime. Flow geometry and fluid clenanup efficiency of highly anisotropic reservoirs and thin beds are strongly related. This implies similar transient effects for anisotropic formations and thin beds, which might trigger the presence of a transition radial flow before achieving a fully developed late-time flow regime.

Notably, mud-filtrate invasion radius affects the early-time regime, permeability anisotropy impacts the transition-time, and the late-time regime is unaffected by near-wellbore reservoir features. Moreover, total porosity and reservoir permeability do not influence the interpretation of the contamination transient analysis via the FCD method. In addition, fluid contamination log-log plot presents a higher resolution than the FCD for the identification of reservoir boundaries and late-time flow regimes. However, this plot lacks a noticeable signature for factors affecting the near-probe flow geometry at early-times, where the FCD offers more detail trends for interpretation purposes.

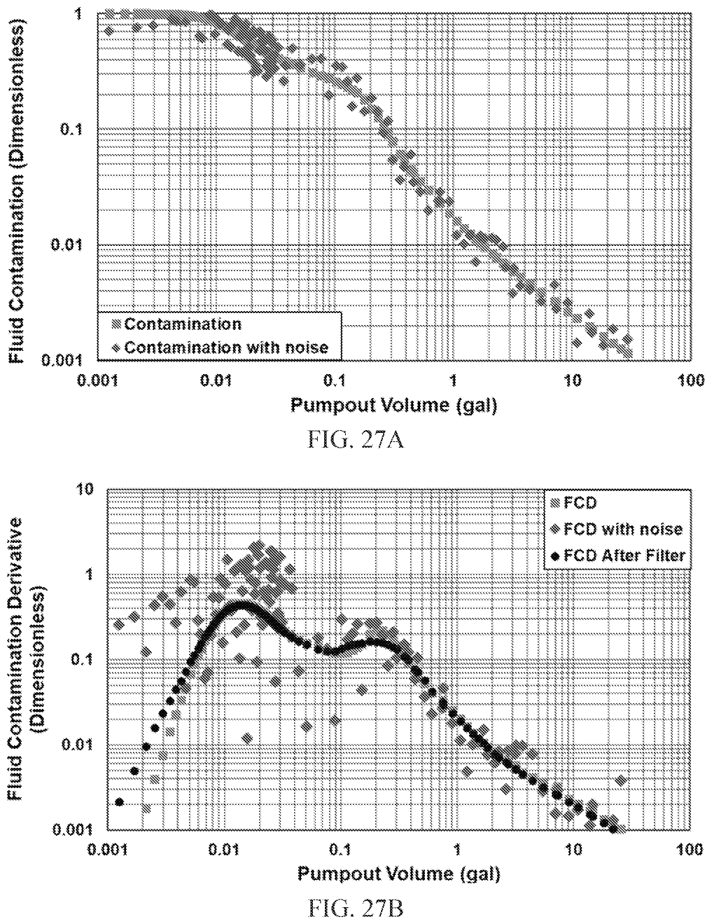

Effect of Noise Present in the Fluid Contamination Measurements. Presence of noise in fluid contamination measurements could be a limitation for the FCD technique because derivative methods tend to amplify the effect of noise. When fluid contamination measurements exhibit large noise-to-signal ratios, the implementation of the FCD method will tend to mask the effect of reservoir properties and emphasize the effect of noise. In order to estimate the impact of noise on the fluid contamination data, 10% zero-mean Gaussian noise was added to the contamination data of the base case. FIG. 27A shows the noisy fluid contamination data together with the noise-free data, while FIG. 27B illustrates the effect of noise in the FCD method with an additive 10% zero-mean Gaussian noise. As mentioned, the noise significantly affects the derivative approach, masking the early- and late-time components of the FCD curve. Consequently, it is extremely important to properly quantify the character of noise and apply a noise filter to the fluid contamination measurements prior to implementing the FCD technique. For this case, median noise filter is used with an adjustable window to enhance the resolution of the contamination curve. Subsequently, the derivative is computed using the center-point technique described in Eq. 5. The combination of the median noise filter in the fluid contamination measurements and the center-point derivative calculation enables high resolution FCD curves and the differentiation of early-time trends and late-time flow regimes.

Discussion. The FCD method and CTA techniques described in this Example are alternative interpretation procedures to detect important reservoir parameters. Late-time regimes include three distinct trends in the log-log plots of fluid contamination and FCD, which are useful to identify reservoir boundaries: spherical flow regime (slope=−⅔), radial flow regime (slope=−3), and boundary effects (slope=−⅓). These well-defined late-time regimes could assist in real-time identification of vertical and radial boundaries, and presence of thin bed or shale laminations. From the sensitivity analysis performed for the identification of reservoir boundaries cases, fully developed radial flow and boundary effects are encountered at 5 ft. from the formation tester probe location. A radius of investigation of the fluid contamination transient and the FCD are also estimated of approximately 10 ft. in the vertical direction and radial directions. However, this radius of investigation might vary based on the combination of other reservoir parameters and operating conditions. The radius of investigation of these techniques is strongly related to filtrate invasion and fluid cleanup mechanisms.

The combination of the fluid contamination log-log plot and the FCD method significantly enhances the interpreter's ability to identify the transient behavior of downhole fluid sampling measurements. Even though the fluid contamination provides higher resolution for the identification of late-time flow regimes, the FCD serves to corroborate these transient trends. In addition, the FCD provides additional information at early-times, where invasion radius, thin beds, reservoir fluid types and permeability anisotropy dominate the flow geometry in the near-probe region. Early-time signatures and late-time flow regimes can be clearly distinguished using the FCD technique, where the change in trends is conspicuous. In conventional fluid cleanup curves, the declining exponential trend makes it difficult to identify the early- and late-time trends, whereas the use of log-log plots and the FCD method increases the definition of the slopes and the identification of flow regimes in the fluid contamination transient.

Qualitative identification of invasion radius and movable fluids are additional advantages of the FCD technique. These key reservoir features can be analyzed during early-time regimes. For instance, the presence of single-phase flow causes a different effect on the FCD from that of multiphase flow. In single-phase flow systems, the FCD curve exhibits a double-hump before transitioning to a spherical flow regime, whereas in multiphase systems the second hump is not observed. This difference between single- and multi-phase flow enhances the ability of the method to distinguish reservoir fluid types and differentiate between hydrocarbon- and water-bearing rocks. Invasion radius can also be estimated under single-phase flow conditions. Consequently, the use of the FCD to identify reservoir fluid type and invasion radius at early-times enables an alternative solution to describe the invaded region.

In practical applications, CTA and the FCD method is useful for assisting in optimizing downhole fluid sampling. The definition of the fluid contamination transient and the identification of early-time features and late-time flow regimes enable reservoir description in real-time and can permit the adjustment of operational parameters during cleanup and sampling. For example, the identification of a deeper invaded region than expected during job planning, would trigger the increase of pumpout rates because of under-estimation of pumpout volumes. Similarly, the detection of radial flow regime at late-times becomes a potential indicator of an ideal moment to finalize cleanup and start the acquisition of fluid samples. Notably, total porosity, reservoir permeability, and permeability anisotropy do not affect the FCD interpretation, thus simplifying the evaluation of the corresponding fluid contamination measurements. The FCD is a transient technique that provides additional degrees of freedom for improved interpretation of fluid contamination measurements, which facilitates the detection of diffusion mechanisms occurring in the near-wellbore region during mud-filtrate cleanup.

Fluid contamination transient analysis is suitable for formation testing applications because it comprises a robust dataset acquired in long periods of fluid cleanup (commonly hours). During downhole fluid sampling, the new interpretation technique described in this Example provides an alternative to PTA, which is limited due to a constraint in pressure pre-test time (a few minutes). CTA does not require additional operational time, extended buildup periods, or extremely large pumpout volumes to observe reservoir fluids trends and flow regimes, thereby saving time and operational costs. Such advantages along with reservoir fluid identification, estimation of invasion radius, and detection of reservoir boundaries emphasize the benefits of the FCD method as an innovative formation evaluation procedure.

Nevertheless, depending on the downhole fluid sampling conditions and fluid cleanup efficiency, the cleanup time and volume may vary. These operational restrictions could limit the radius of investigation of the fluid contamination transient and obscure the observation of flow regimes. Fluid cleanup time and pumpout operating parameters are variable and depend on each case. Therefore, a practical time length or pumpout volume for the application of CTA techniques cannot be easily defined. The methods presented in this Example do not encourage to extend fluid cleanup times to achieve certain flow regimes. On the contrary, these approaches provide alternatives to increase fluid cleanup efficiency by understanding flow regimes. Obtaining low contamination samples and avoiding operational risks are the main objectives of downhole fluid sampling, whereas CTA and the FCD method provide an additional benefit for formation-testing applications.

For implementation purposes, early-time effects, such as those caused by a different phase-flow between the reservoir fluid and mud-filtrate, invasion radius, and permeability anisotropy can be detected employing the methods described herein. Similar as other pressure transient analysis advances, further development of techniques involving type-curves, fluid cleanup models, and dimensionless analysis can serve for the quantification of invasion radius and permeability anisotropy. Likewise, if fluid contamination is sufficiently low, the late-time flow regimes can be identified. If no-flow boundaries are in the proximity of the formation tester probe and fluid geometry disturbance attains formation limits, boundary effects and radial flow regimes can not only be identified, but potentially quantified using any fluid contamination model with the correct time or volume power-law function. The quantification of these values for spherical flow regime (−⅔), radial flow regime (−3), and boundary effects (−⅓) enable the modification of analytical models and an accurate estimation of fluid contamination in real-time.

Conclusions. Transient techniques based on downhole fluid contamination measurements and its derivative function are described in terms of pumpout volume and cleanup time. Fluid contamination transient analysis and the strong relation between near-probe flow geometry and transient responses for formation-testing applications are also described. Advantageous contributions of this Example include:

Fluid contamination and FCD curves enable the visual identification of spherical flow regime, radial flow regime, and boundary effects, which are denoted by late-time log-log slopes of −⅔, −3, and −⅓, respectively. These power-law parameters could be used as inputs in analytical models for real-time estimation of fluid contamination and characterization of the invaded region.

Thin bed or shale laminations, phase-flow differences between mud-filtrate and clean reservoir fluid, invasion radius, and permeability anisotropy affect the early-time signature of the FCD, which permits the detection of these parameters in the near-wellbore.

The evident differences observed in the sensitivity analysis for the various numerical-simulations cases could serve as groundwork for the development of novel transient techniques, such as type-curves, dimensionless analysis, and new mathematical models.

It is also found that all the benefits of the FCD method are attainable independently of the underlying petrophysical properties, such as porosity and permeability.

A suitable value for the smoothing factor, X, is shown in a range from 0 to 0.5. If the FCD noise-to-signal-ratio is high at X=0.5, a noise median filter on the fluid contamination measurements before implementing the FCD method can be used.

Nomenclature
C=fluid contamination, dimensionless
V=pumpout volume, cm³ (gal)
t=time, s
φ=fluid property measured with formation testers
X=smoothing factor
h=formation thickness, m (ft)
Re=radial reservoir extension, m (ft)
ø=total porosity, dimensionless
k=permeability, and
kv/kh=vertical to horizontal permeability ratio, dimensionless

Subscripts
f=reservoir fluid
mf=mud-filtrate
i=center point
L=location before the center point
R=location after the center point

Acronyms
PTA=Pressure Transient Analysis
CTA=Contamination Transient Analysis
FCD=Fluid Contamination Derivative
WBM=Water-Base Mud

Figure captions for Example 2. FIG. 17: Numerical simulation model showing the probe grid refinement and a top view of the near-wellbore zone during fluid cleanup simulation. Green color depicts the probe effective flow area, while red blocks identify probe seals.

FIG. 18: Base case single-phase flow: log-log plot of fluid contamination and FCD. Red squares identify the fluid contamination data, black dots identify the fluid contamination derivative, and the blue straight lines provide a reference slope equal to −⅔ at late-times.

FIG. 19A and FIG. 19B: Multiphase flow case: log-log plot of (FIG. 19A) fluid contamination, and (FIG. 19B) FCD. Black dots identify the base case for single-phase flow, red squares identify the base case for multiphase flow, and the blue straight lines provide a reference slope equal to −⅔ at late-times.

FIG. 20: Smoothing factor case: Sensitivity analysis for noise reduction and over smoothing evaluation in the application of the FCD. Black dots identify the base case with X=0, red squares identify the base case with X=0.2, and blue triangles identify the base case with X=0.5.

FIG. 21A and FIG. 21B: Radial boundaries case: log-log plot of (FIG. 21A) fluid contamination, and (FIG. 21B) FCD. Black dots identify the base case, red squares identify the case with a radial boundary located at 5 ft. from the wellbore, blue triangles identify the case with a radial boundary located at 10 ft. from the wellbore, green rhombuses identify the case with a radial boundary located at 15 ft. from the wellbore, and the blue straight lines provide a reference slope equal to −⅓ at late-times.

FIG. 22A and FIG. 22B: Vertical boundaries case: log-log plot of (FIG. 22A) fluid contamination, and (FIG. 22B) FCD. Black dots identify the base case, red squares identify the case for a reservoir model with a formation thickness of 5 ft., blue triangles identify the case for a reservoir model with a formation thickness of 10 ft., green rhombuses identify the case for a reservoir model with a formation thickness of 20 ft., and the blue straight lines provide a reference slope equal to −3 at late-times.

FIG. 23A and FIG. 23B: Thinly-laminations case: log-log plot of (FIG. 23A) fluid contamination, and (FIG. 23B) FCD. Black dots identify the base case, red squares identify the case with 3 in thinly laminated formation, blue triangles identify the case with 4 in thinly laminated formation, and green rhombuses identify the case with 6 in thinly laminated formation.

FIG. 24A and FIG. 24B: Mud-filtrate invasion radius case: log-log plot of (FIG. 24A) fluid contamination, and (FIG. 24B) FCD. Black dots identify the base case, red squares identify the case with a mud-filtrate invasion radius of 12 in., and blue triangles identify the case with a mud-filtrate invasion radius of 24 in.

FIG. 25A and FIG. 25B: Reservoir properties case: log-log plot of (FIG. 25A) fluid contamination, and (FIG. 25B) FCD. Black dots identify the base case, red squares identify the case for a reservoir model with a total porosity of 0.05 (5%), blue triangles identify the case for a reservoir model with a total porosity of 0.35 (35%), green rhombuses identify the case with a reservoir permeability of 8 md, and purple dash lines identify the case with a reservoir permeability of 800 md.

FIG. 26A and FIG. 26B: Permeability anisotropy case: log-log plot of (FIG. 26A) fluid contamination, and (FIG. 26B) FCD. Black dots identify the base case homogeneous isotropic reservoir, red squares identify the case with a vertical-permeability-to-horizontal-permeability ratio of 0.1, blue triangles identify the case with a vertical-to-horizontal-permeability ratio of 0.2, and green rhombuses identify the case with a vertical-to-horizontal-permeability ratio of 0.4.

FIG. 27A and FIG. 27B: Gaussian noise case: log-log plot of (FIG. 27A) fluid contamination, and (FIG. 27B) FCD, with 10% zero-mean Gaussian noise for the base case numerical simulations for single-phase flow. Red squares identify the base case without noise, blue rhombuses identify the case with 10% zero-mean Gaussian noise, and black dots identify the FCD curve after applying the noise median filter on the fluid contamination measurements.

### Example 3: New Contamination Cleanup Inversion Methods and Interpretation Model

During real time cleanup operations, change in contamination is continuously measured to determine fluid sample quality. However, accurate assessment of the estimated time or pump-out volume required to achieve the contamination target is extremely challenging due to multiple factors impacting the fluid cleanup trend. This contamination target value varies depending on the fluid sample quality necessary to perform laboratory tests, such as Pressure-Volume-Temperature (PVT) measurements, fluid characterization or chromatography, and asphaltene offset studies. Therefore, estimating fluid cleanup pump-out volume or time and the uncertainty in the quality of the sample are the major problems when dealing with the acquisition of bottom-hole fluid samples. Contamination cleanup decay can be modeled as a summation of exponentials due to the diffusive characteristics of fluid pump-out and the transient evolution of the invaded front until attaining the optimal conditions to sample. The time and cumulative pump-out volume evolution of fluid contamination responds to diffusion mechanisms comparable to the magnetization decay of protons in Nuclear Magnetic Resonance (NMR). A Nuclear magnetic resonance (NMR) physics uses magnetic fields to excite protons and allow for relaxation and diffusion to obtain the magnetic decay, which exhibits an exponential decay. The properties of pore fluids that affect the NMR echo trains are the hydrogen index (HI), the longitudinal relaxation time (T₁), the transverse relaxation time (T₂) and the diffusivity (D). NMR inversion relates the magnetization decay as a summation of exponentials following these mathematical expressions for the T₁ and T₂ distributions, respectively:

$$\begin{matrix}{M}_{( t )} = Σ {M}_{0} [ 1 - {e}^{- t / {T}_{1}} ] & ( 1 ) \\ {M}_{( t )} = Σ {M}_{0} {e}^{- t / {T}_{2}} & ( 2 )\end{matrix}$$

where t is the time that protons are exposed to the magnetic field, M₍ₜ₎ is the magnitude of magnetization as a function of time, M₀ is the maximum magnetization, T₁ is the time at which the magnetization attains 63% of its final value, and T₂ is the transverse relaxation time. On the other hand, Pressure Transient Analysis (PTA) employs various techniques to characterize the reservoir complexity through the evaluation of transient diffusive trends. For instance, the pressure derivative method can be accurately used to identify flow regimes, reservoir geometry and boundaries. Therefore diffusion physics of the pressure transient allows the use of a similar exponential-based model as the model presented for NMR. The inversion of the pressure buildup exponential trend and construction of a distribution analogous to the T₁ distribution could be another alternative to PTA techniques for flow regimes identification and reliably estimation of reservoir and near-wellbore properties.

Similarly to PTA, Contamination Transient Analysis (CTA) permits identification of flow regimes during bottom-hole fluid cleanup and sampling operations performed with formation testers. The flow regimes identification and trends serve to estimate reservoir limits, such as formation thickness and faults, presence of shale laminations, permeability anisotropy and depth of mud-filtrate invasion. The assessment of these properties are the key to optimize fluid cleanup and sampling times. Likewise, the application of the inversion method to the fluid contamination decay provides a real-time match of the cleanup data and the ability to estimate the volume and time required to reach the desired contamination target. This Example advantageously provides a novel model to reproduce the fluid contamination cleanup decay in real time using a summation of exponentials inversion technique. This Example presents the methodology used to develop the new model, and its results using synthetic and field data. A discussion of the results and its implications are provided.

Methodology. The model represents the fluid contamination cleanup decay and the pressure buildup as a summation of exponentials. The methodology implemented in this Example performs a non-linear inversion technique using the Tikhonov regularization approach on formation testing data for pressure pretests and fluid cleanup and sampling operations. This inversion approach provides a model based on summation of exponentials to match the data and generate an equation for pressure buildup and fluid contamination, respectively:

$$\begin{matrix}Δ {P}_{( t )} = Σ {A}_{0} [ 1 - {e}^{- t / T} ] & ( 3 ) \\ {C}_{( vol )} = Σ {A}_{0} {e}^{- vol / PV} & ( 4 )\end{matrix}$$

where t is the time, ΔP₍ₜ₎ is the change in time of the pressure buildup, vol is the pump-out volume, C₍ᵥₒₗ₎ is the contamination decay as a function of pump-out volume, A₀ is the maximum amplitude, and T and PV are the time constant and volume constant used to normalize and generate the distributions. These models are validated using the pressure buildup as a benchmark with multiple numerical simulation scenarios and sensitivities for radial flow and reservoir boundaries identification, and the impact of wellbore storage, skin, variable drawdown rates and presence of noise. The applicability of the method in fluid cleanup and sampling operations is tested with synthetic examples generated with numerical simulation. Seven reservoir simulation cases are developed to obtain contamination data: 1) base case: homogeneous isotropic reservoir, 2) formation thickness, 3) laminated formations, 4) geological faults, 5) mud-filtrate invasion 6) reservoir properties, and 7) permeability anisotropy. All these cases for pressure buildup and contamination data are compared versus the transient analysis techniques of the pressure derivative and the reciprocal contamination derivative, respectively. The model output comprises a match for the complete curve of the contamination decay, independently of the transient trends changes, and the pump-out volume and time distributions, which are useful to recognize the trend changes in real time in order to estimate the volume and time required to achieve the required contamination target. In addition, these distributions may be useful to quantify diverse reservoir properties and flowrate conditions.

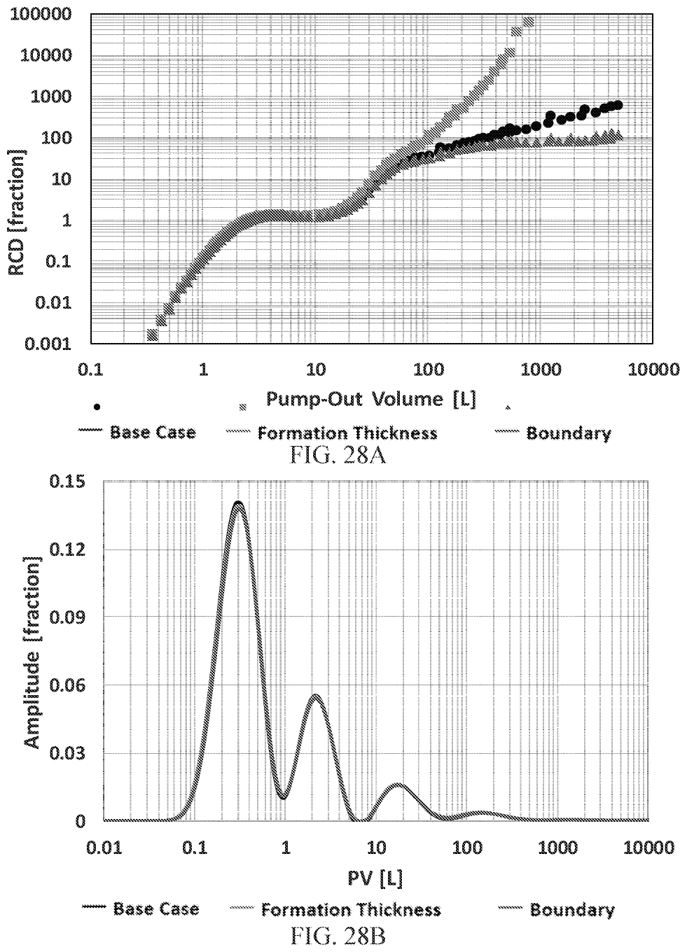

Fluid Contamination Cleanup. After validating the inversion method using the pressure derivative and transient analysis as benchmark, this Example introduces the approach for modeling fluid contamination cleanup measurements. The main difference with respect to pressure data is the possibility to apply the fluid Pump-out Volume (PV) instead of time as a variable in the analysis and distributions. Pump-out volume is recommended for this type of data because it normalizes the impact of flowrate variability in formation testing operations. First, the Reciprocal Contamination Derivative (RCD) is shown to be useful in CTA applications to identify flow regimes and reservoir boundaries. FIG. 28A and FIG. 28B present the comparison between the RCD and the PV distribution. As observed, the RCD method has a higher resolution for identifying flow regimes at late-times. The PV distribution for the base case of a homogeneous isotropic reservoir with a formation thickness of 100 ft. and a radial boundary of 400 ft. looks similar in the case with only 5 ft. of vertical and radial limits.

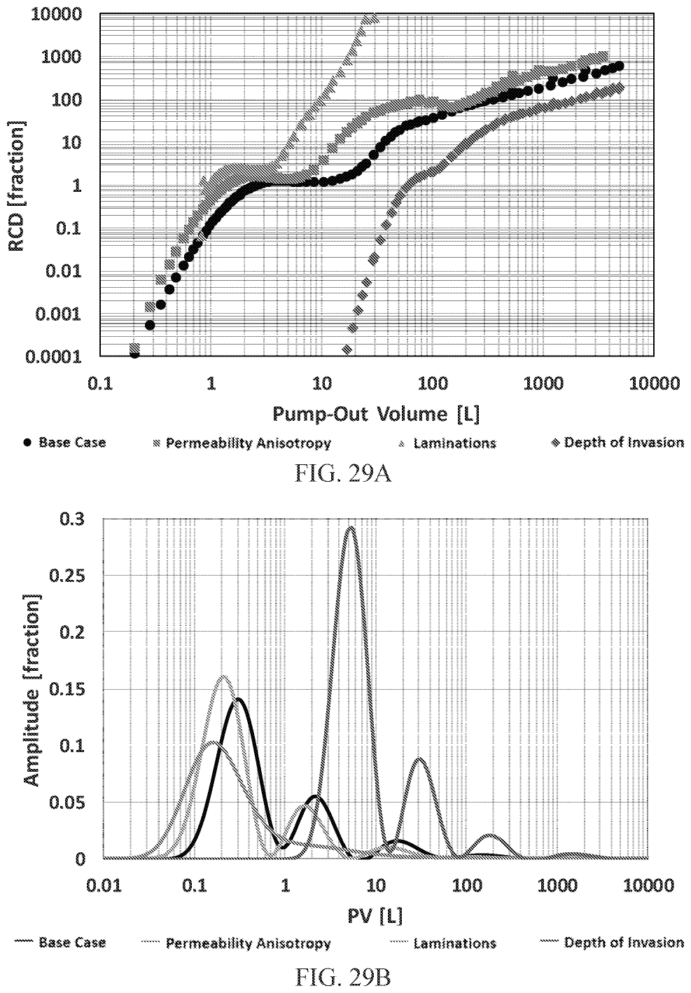

Since the PV distribution has limitation to represent late-time flow regimes in the contamination transient, simulation cases were performed to observe the PV distributions for changes in the permeability anisotropy, the presence of thinly laminated formations and the influence of the invasion depth. These PV distribution cases were compared with the RCD (FIG. 29A and FIG. 29B), and interesting changes were noted in the PV distributions in terms of peaks location and amplitudes. For the case of permeability anisotropy (kv/kh=0.1), the PV maximum locations are perceived to shift to the left in the PV domain (x-axis) with respect to the base case (isotropic reservoir). This observation is consistent with the impact of permeability anisotropy in cleanup efficiency because fluids move faster in the radial direction than in the vertical direction, which generates a natural focused effect and allows to withdraw fluids more efficiently. On the other hand, if the depth of invasion is increased from 6 inches for the base case to 24 inches to the deep invasion case, the PV distribution is shifted to the right in the PV axis as expected. A higher invasion depth would require a longer pump-out volume during cleanup. Therefore, a delay may be observed in both the PV distribution and the derivative approach. However, the first peak in the PV distribution can be detected much faster than the trend on the RCD curve. For the PV distribution, the maximum PV is attained at 5 liters, whereas the RCD curve begins to show its trend after the fluid pumped volume is above 100 liters. In addition, the first peak amplitude on the PV distribution is significantly higher for the deeply invaded case, indicating that the invasion volume could be estimated with the quantification of this first peak. Likewise, the thinly laminated case (3 inches sandstone-shale laminations) presents a completely different PV distribution when compared with the base case (pure sandstone). In this case, only one dominant peak or one dominant exponential is evident in the PV distribution. This dominant peak has its maximum at lower PV than the base case, which is consistent with the effect of laminations in the performance of fluid cleanup. Therefore, the use of the PV distribution is useful as an alternative or complementary approach to the RCD method for characterizing laminated reservoirs. In general, the PV distribution can quantify near wellbore properties and improve the understanding of the invaded region.

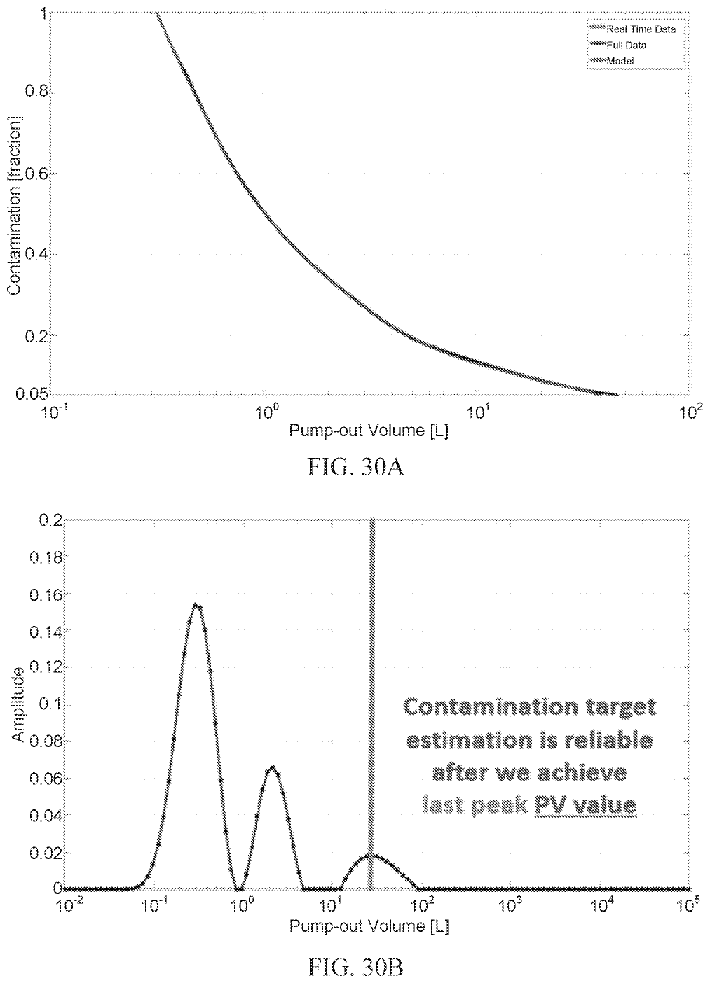

Moreover, this inversion method proved to be extremely useful in estimating the fluid contamination target. The contamination measurements are matched in real time and the PV distribution is generated at the same time. With this information, a model is generated using equation (4) and the cleanup trend is estimated until the contamination target is achieved. Indeed, the understanding of this inversion method indicates that each peak in the PV distribution obeys to a change in trend or to a dominant exponential. When the real time pump-out volume achieves the maximum amplitude of the last peak in the PV distribution (FIG. 30A and FIG. 30B), accurate prediction of the cumulative pumped volume required to acquire non-contaminated fluid samples can be achieved. For the base case, the contamination target is achieved after pumping around 45 liters, and this value can be reliably predicted since a cumulative pumped volume of 25 liters is achieved. As a summary, the workflow to assess the contamination target in real time is: 1) Apply the inversion technique at each data step; 2) monitor the presence of peaks until detecting the last peak in the PV distribution (last dominant cleanup trend); 3) when pump-out data attains the last peak PV value, use the model to predict the cumulative pumped volume required to achieve the contamination target.

Conclusions. Ultimately, the diffusive and transient nature of the contamination decay and the pressure buildup allow modeling pressure and fluid cleanup measurements acquired with formation testers as a summation of exponentials following equations (3) and (4), respectively. Furthermore, this novel inversion and model allows to generate time T distributions and pump-out PV distributions, which serve as alternative to pressure transient analysis and contamination transient analysis techniques for reservoir characterization purposes. Flow regimes identification, detection of reservoir boundaries and qualitative estimation of near wellbore features are possible due to the evaluation of the mentioned distributions. In addition, variable flowrates and presence of noise does not affect the functionality of the model to match formation testing measurements and to perform the evaluation of the T and PV distributions. As a result, this technique can be used to match contamination measurements in real time formation testing applications allowing to accurately predict the desire target to acquire non-contaminated fluid samples. An advantage of this methodology is that it is now possible to estimate this with sufficient time to improve decision making to optimize cleanup efficiency. Therefore, this novel inversion and model is an effective and reliable method to estimate contamination target in real time fluid cleanup and sampling operations.

Figure captions for Example 3. FIG. 28A and FIG. 28B: Comparison of the base case and reservoir limits cases (FIG. 28A) RCD, and (FIG. 28B) PV distribution.

FIG. 29A and FIG. 29B: Comparison of the base case with near wellbore features cases (FIG. 29A) RCD, and (FIG. 29B) PV distribution.

FIG. 30A and FIG. 30B: Real time contamination target estimation for the base case (FIG. 30A) cleanup curve, and (FIG. 30B) PV distribution.

### REFERENCES

Akram, A. H., Halford, F. R., and Fitzpatrick, A. J. (1999). A model to predict wireline formation tester sample contamination. SPE Reservoir Evaluation & Engineering, 2 (06), 499-505. Society of Petroleum Engineers. doi:10.2118/59559-PA.
Arps, J. J. (1945). Analysis of decline curves. Transactions of the AIME, 160 (01), 228-247. Society of Petroleum Engineers. doi:10.2118/945228-G.
Agarwal, R. G., Gardner, D. C., Kleinsteiber, S. W., and Fussell, D. D. (1999). Analyzing well production data using combined-type-curve and decline-curve analysis concepts. SPE Reservoir Evaluation & Engineering 2 (05), 478-486. Society of Petroleum Engineers. doi:10.2118/57916-PA.
Bourdet, D., Ayoub, J. A., and Pirard, Y. M. (1989). Use of pressure derivative in well test interpretation. SPE Formation Evaluation, 4 (02), 293-302. doi:10.2118/12777-PA.
Carpenter, C. (2015). Pressure-transient analysis as an element of permanent reservoir monitoring. Journal of Petroleum Technology, 67 (09). Society of Petroleum Engineers. doi:10.2118/0915-0111-JPT.
Chatas, A. T. (1966). Unsteady spherical flow in petroleum reservoirs. Society of Petroleum Engineers Journal 6 (02), 102-114, doi:10.2118/1305-PA.
Fetkovich, M. J. (1980). Decline curve analysis using type curves. Journal of Petroleum Technology, 32 (06), 1065-1077. Society of Petroleum Engineers. doi:10.2118/4629-PA.
Galvan-Sanchez, F., Pragt, J., Vieitez, C., Patil, T., and Ganjoo, V. (2016). The effects of repeated interruptions during the clean-up cycle in a challenging logging-while-drilling sampling operation. SPWLA 57th Annual Logging Symposium, Reykjavik, Iceland, 25-29 June. Society of Petrophysicists and Well-Log Analysts.
Gelvez, C., Torres-Verdin, C., Se, Y., et al. (2019). What if there was a better formation-testing probe? A case study on optimizing flow geometry. SPWLA 60th Annual Logging Symposium, The Woodlands, Tex., USA, 15-19 June. Society of Petrophysicists and Well-Log Analysts. doi: 10.30632/T60ALS-2019_L.
Hammond, P. S. (1991). One- and two-phase flow during fluid sampling by a wireline tool. Transport in Porous Media, 6, 299-330.
Hadibeik, A., Proett, M., Torres-Verdin, C., Sepehrnoori, K., and Angeles, R. (2009). Wireline and while-drilling formation-tester sampling with oval, focused, and conventional probe types in the presence of water- and oil-base mud-filtrate invasion in deviated wells. SPWLA 50th Annual Logging Symposium, The Woodlands, Tex., USA, 21-24 June. Society of Petrophysicists and Well-Log Analysts.
Hammond, P. S. (1991). One- and two-phase flow during fluid sampling by a wireline tool. Transport in Porous Media, 6, 299-330.
House, J., Michel, F., Penna, P., and Alexander, R. (2015). Advanced reservoir fluid characterization using logging-while-drilling: a deepwater case study. SPWLA 56th Annual Logging Symposium, Long Beach, Calif., USA, 18-22 July. Society of Petrophysicists and Well-Log Analysts.
Kemp, K., Elshahawi, H., Megat, A., Tollefsen, E., Kok, J., and Fey, S. (2011). Analyzing the critical effects of time on station. SPWLA 52nd Annual Logging Symposium, Long Beach, Calif., USA, 18-22 July. Society of Petrophysicists and Well-Log Analysts.
Kristensen, M., Ayan, C., Chang, Y., Lee, R., Gisolf, A., Leonard, J., Corre, P.-Y., and Dumont, H. (2014). Flow modeling and comparative analysis for a new generation of wireline formation tester modules. International Petroleum Technology Conference, Doha, Qatar, 20-22 January. International Petroleum Technology Conference. doi:10.2523/IPTC-17385-MS.
Kristensen, M., Chugunov, N., Gisolf, A., Biagi, M., and Dubost, F. (2019). Real-time formation evaluation and contamination prediction through inversion of downhole fluid-sampling measurements. SPE Reservoir Evaluation & Engineering 22 (02), 531-547. Society of Petroleum Engineers. doi:10.2118/187432-PA.
Kristensen, M., Ayan, C., Chang, Y., et al. (2014). Flow modeling and comparative analysis for a new generation of wireline formation tester modules. International Petroleum Technology Conference, Doha, Qatar, 20-22 January. International Petroleum Technology Conference. doi:10.2523/IPTC-17385-MS.
Lee, R., Chen, L., Gisolf, A., Zuo, J. Y., Meyer, J. C., and Campbell, T. (2016). Real-time formation testing focused-sampling contamination estimation. SPWLA 57th Annual Logging Symposium, Reykjavik, Iceland, 25-29 June. Society of Petrophysicists and Well-Log Analysts.
Liu, N. and Oliver, D. S. (2003). Evaluation of Monte Carlo methods for assessing uncertainty. SPE Journal, 8 (02), 188-195. Society of Petroleum Engineers. doi:10.2118/84936-PA.
Malik, M., Torres-Verdin, C., Sepehrnoori, K., et al. (2009). Comparison of wireline formation-tester sampling with focused and conventional probes in the presence of oil-base mud-filtrate invasion. Petrophysics, 50 (05), 1-20. Society of Petrophysicists and Well-Log Analysts.
Moran, J. H. and Finklea, E. E. (1962). Theoretical analysis of pressure phenomena associated with the wireline formation tester. Journal of Petroleum Technology 14 (08), 899-908. Society of Petroleum Engineers. doi: 10.2118/177-PA.
Mullins, O. C. and Schroer, J. (2000). Real-time determination of filtrate contamination during openhole wireline sampling by optical spectroscopy. SPE Annual Technical Conference and Exhibition, Dallas, Tex., 1-4 October. Society of Petroleum Engineers. doi:10.2118/63071-MS.
Onur, M., Hegeman, P. S., and Kuchuk, F. J. (2004). Pressure/pressure convolution analysis of multiprobe and packer-probe wireline formation tester data. SPE Reservoir Evaluation & Engineering 7 (05), 351-364. Society of Petroleum Engineers. doi:10.2118/88994-PA.
Prasad, T., Castillo, H. C., and Elshahawi, H. (2012). Effective mitigation of tool sticking risk in formation testing and fluid sampling operations. SPE Annual Technical Conference and Exhibition, San Antonio, Tex., USA, 8-10 October. Society of Petroleum Engineers. doi:10.2118/159246-MS.
Proett, M., Welshans, D., Sherrill, K., Wilson, J., House, J., Shokeir, R., and Solbakk, T. (2010). Formation testing goes back to the future. SPWLA 51st Annual Logging Symposium, Perth, Australia, 19-23 June. Society of Petrophysicists and Well-Log Analysts.
Zhao, Y. and Reynolds, A. C. (2009). Estimation and removal of tidal effects from pressure data. SPE Journal, 14 (01), 144-152. Society of Petroleum Engineers. doi:10.2118/103253-PA.
Zuo, J. Y., Gisolf, A., Dumont, H., et al. (2015) A breakthrough in accurate downhole fluid sample contamination prediction in real time. Petrophysics 56 (03), 251-265. Society of Petrophysicists and Well-Log Analysts.
Zuo, J. Y., Gisolf, A., Wang, K., Dubost, F., Pfeiffer, T., Dumont, H., Mishra, V., Chen, L., Agarwal, A., Ayan, C., and Mullins, O. C. (2016) Quantitative mixing rules for downhole oil-based mud contamination monitoring in real time using multiple sensors. Journal of Petroleum Science and Engineering, 137, 214-226.

FIG. 31 is a diagram illustrating an example architecture 3100 for implementing an automated formation condition estimation technique, in accordance with at least one embodiment. In architecture 3100, one or more users 3102 (e.g., customers, users, consumers, etc.) may utilize user computing devices 3104(1)-(N) (collectively, user devices 3104) to access a browser application 3106 or a user interface (UI), optionally accessible through the browser application 3106, via one or more networks 3108. The “browser application” 3106 can be any browser control or native application that can access and display a network page or other information such as a user interface of a native software application for enabling the selection or interaction of content. A native software application may include an application or program that has been developed for use on a particular platform (such as an operating system) or a particular device (such as a particular type of mobile device or user device 3104). In embodiments, the user device 3104 may include one or more components for enabling the user 3102 to interact with the browser application 3106.

The user devices 3104 may include at least one memory 3110 and one or more processing units or processors 3112. The memory 3110 may store program instructions that are loadable and executable on the processor(s) 3112, as well as data generated during the execution of these programs. Depending on the configuration and type of the user devices 3104, the memory 3110 may be volatile (such as random access memory (RAM)) and/or non-volatile (such as read-only memory (ROM), flash memory, etc.). The user devices 3104 may also include additional removable storage and/or non-removable storage including, but not limited to, magnetic storage, optical disks, and/or tape storage. The disk drives and their associated non-transitory computer-readable media may provide non-volatile storage of computer-readable instructions, data structures, program modules, and other data for the user devices 3104. In some implementations, the memory 3110 may include multiple different types of memory, such as static random access memory (SRAM), dynamic random access memory (DRAM), or ROM.

Turning to the contents of the memory 3110 in more detail, the memory 3110 may include an operating system and one or more application programs or services for implementing the techniques disclosed herein. Additionally, the memory 3110 may include one or more modules for implementing the techniques described herein including a content validation module 3130.

The architecture 3100 may also include one or more service provider computers 3114 that may, in some examples, provide computing resources such as, but not limited to, client entities, low latency data storage, durable data store, data access, management, virtualization, hosted computing environment or “cloud-based” solutions, electronic content performance management, etc. The service provider computers 3114 may be carried by or be an example of the testing tool described herein with reference to FIGS. 1-2 and throughout the disclosure.

In some examples, the networks 3108 may include any one or a combination of many different types of networks, such as cable networks, the Internet, wireless networks, cellular networks, and other private and/or public networks. While the illustrated examples represents the users 3102 communicating with the service provider computers 3114 over the networks 3108, the described techniques may equally apply in instances where the users 3102 interact with the one or more service provider computers 3114 via the one or more user devices 3104 over a landline phone, via a kiosk, or in any other manner. It is also noted that the described techniques may apply in other client/server arrangements (e.g., wireline communication, etc.), as well as in non-client/server arrangements (e.g., locally stored applications, peer-to-peer arrangements, etc.).

The one or more service provider computers 3114 may be any type of computing devices such as, but not limited to, a mobile phone, a smart phone, a personal digital assistant (PDA), a laptop computer, a desktop computer, a server computer, a thin-client device, a tablet PC, etc. Additionally, it should be noted that in some embodiments, the one or more service provider computers 3114 may be executed by one or more virtual machines implemented in a hosted computing environment. The hosted computing environment may include one or more rapidly provisioned and released computing resources, which computing resources may include computing, networking, and/or storage devices. A hosted computing environment may also be referred to as a cloud computing environment or distributed computing environment. In some examples, the one or more service provider computers 3114 may be in communication with the user device 3104 via the networks 3108, or via other network connections. The one or more service provider computers 3114 may include one or more servers, perhaps arranged in a cluster or as individual servers not associated with one another.

In one illustrative configuration, the one or more service provider computers 3114 may include at least one memory 3116 and one or more processing units or processor(s) 3118. The processor(s) 3118 may be implemented as appropriate in hardware, computer-executable instructions, firmware, or combination thereof. Computer-executable instruction or firmware implementations of the processor(s) 3118 may include computer-executable or machine-executable instructions written in any suitable programming language to perform the various functions described when executed by a hardware computing device, such as a processor. The memory 3116 may store program instructions that are loadable and executable on the processor(s) 3118, as well as data generated during the execution of these programs. Depending on the configuration and type of the one or more service provider computers 3114, the memory 3116 may be volatile (such as RAM) and/or non-volatile (such as ROM, flash memory, etc.). The one or more service provider computers 3114 or servers may also include additional storage 3120, which may include removable storage and/or non-removable storage. The additional storage 3120 may include, but is not limited to, magnetic storage, optical disks and/or tape storage. The disk drives and their associated computer-readable media may provide non-volatile storage of computer-readable instructions, data structures, program modules, and other data for the computing devices. In some implementations, the memory 3116 may include multiple different types of memory, such as SRAM, DRAM, or ROM.

The memory 3116, the additional storage 3120, both removable and non-removable, are all examples of non-transitory computer-readable storage media. For example, computer-readable storage media may include volatile or non-volatile, removable or non-removable media implemented in any method or technology for storage of information such as computer-readable instructions, data structures, program modules, or other data. The memory 3116 and the additional storage 3120 are all examples of non-transitory computer storage media. Additional types of non-transitory computer storage media that may be present in the one or more service provider computers 3114 may include, but are not limited to, PRAM, SRAM, DRAM, RAM, ROM, EEPROM, flash memory or other memory technology, CD-ROM, DVD, or other optical storage, magnetic cassettes, magnetic tape, magnetic disk storage or other magnetic storage devices, or any other medium which can be used to store the desired information and which can be accessed by the one or more service provider computers 3114. Combinations of any of the above should also be included within the scope of non-transitory computer-readable media.

The one or more service provider computers 3114 may also contain communication connection interface(s) 3122 that allow the one or more service provider computers 3114 to communicate with a data store, another computing device or server, user terminals, and/or other devices on the networks 3108. The one or more service provider computers 3114 may also include I/O device(s) 3124, such as a keyboard, a mouse, a pen, a voice input device, a touch input device, a display, speakers, a printer, etc.

Turning to the contents of the memory 3116 in more detail, the memory 3116 may include an operating system 3126, one or more data stores 3128, and/or one or more application programs or services for implementing the techniques disclosed herein including the formation condition determination module 3130. In accordance with at least one embodiment, the formation condition determination module 3130 may be configured, using a formation testing tool, to obtain a sampled fluid from a formation according to a set of sampling parameters and to analyze the sampled fluid to identify a set of fluid parameters for the sampled fluid, and, using a numerical model, determine a formation condition. In accordance with at least one embodiment, the service provider computers 3114 and formation condition determination module 3130 may be configured to store data associated with determination operations (e.g., datasets detailing model outputs and measurements) in data store 3128 or, via networks 3108, to distributed data storage systems (e.g., cloud storage systems).

### Illustrative Aspects

As used below, any reference to a series of aspects (e.g., “Aspects 1-4”) or non-enumerated group of aspects (e.g., “any previous aspect” or “any previous or subsequent aspect”) is to be understood as a reference to each of those aspects disjunctively (e.g., “Aspects 1-4” is to be understood as “Aspects 1, 2, 3, or 4”).

Aspect 1 is a method comprising: using a formation testing tool to obtain a sampled fluid from a formation according to a set of sampling parameters; using the formation testing tool to analyze the sampled fluid to identify a set of fluid parameters for the sampled fluid; and using a numerical model to determine a formation condition, wherein inputs for the numerical model include the set of sampling parameters and the set of fluid parameters.

Aspect 2 is the method of any previous or subsequent aspect, further comprising repeating one or more times: using the numerical model to generate an updated set of sampling parameters; using the formation testing tool to obtain additional sampled fluid from the formation according to the updated set of sampling parameters; using the formation testing tool to analyze the additional sampled fluid to identify an updated set of fluid parameters for the additional sampled fluid; and using the numerical model to generate an updated formation condition, wherein inputs for the numerical model further include the updated set of sampling parameters and the updated set of fluid parameters.

Aspect 3 is the method of any previous or subsequent aspect, wherein inputs for the numerical model further include one or more of historical fluid parameters for fluid sampled from the formation, simulated fluid parameters for fluid sampled from the formation, historical fluid parameters for fluid sampled from a different formation, and simulated fluid parameters for fluid sampled from the different formation.

Aspect 4 is the method of any previous or subsequent aspect, wherein the set of sampling parameters comprises sampling conditions associated with obtaining the sampled fluid.

Aspect 5 is the method of any previous or subsequent aspect, wherein the set of sampling parameters comprises a drawdown rate used for sampling fluid from the formation, a drawdown pressure used for sampling fluid from the formation, an injection rate for injecting fluid from the formation testing tool into the formation during sampling, a buildup pressure measured after sealing the testing tool, or a characteristic dimension of the formation testing tool.

Aspect 6 is the method of any previous or subsequent aspect, wherein the set of sampling parameters further comprise a pulse sequence, the pulse sequence including one or more modifications to the drawdown rate, the drawdown pressure, the injection rate, or the buildup pressure in an ordered sequence during sampling fluid from the formation.

Aspect 7 is the method of any previous or subsequent aspect, wherein the set of fluid parameters for the sampled fluid comprises analytical results associated with evaluating the sampled fluid.

Aspect 8 is the method of any previous or subsequent aspect, wherein the set of fluid parameters for the sampled fluid comprises at least one of a mass density for the sampled fluid, a fluid viscosity for the sampled fluid, a fluid resistivity for the sampled fluid, a formation pressure, an estimated formation pressure, an optical density for the sampled fluid, a level of contamination for the sampled fluid, a speed of sound in the sampled fluid, a gas-to-liquid ratio for the sampled fluid, a composition of the sample fluid, or a formation volume factor for the sampled fluid.

Aspect 9 is the method of any previous or subsequent aspect, wherein fluid parameters of the set of fluid parameters are determined as a function of time or a function of pumpout volume.

Aspect 10 is the method of any previous or subsequent aspect, wherein the formation condition comprises one or more of: predicted contamination for additional fluid sampled from the formation as a function of time or pumpout volume; a predicted time at which additional fluid sampled from the formation contains a target amount or less of contamination; a predicted pumpout volume at which additional fluid sampled from the formation contains a target amount or less of contamination; or a predicted lowest level of contamination for additional fluid sampled from the formation.

Aspect 11 is the method of any previous or subsequent aspect, further comprising: generating a notification providing the formation condition.

Aspect 12 is the method of any previous or subsequent aspect, wherein the notification includes one or more of an indication of a predicted lowest level of contamination for additional fluid sampled from the formation, or a predicted duration until additional fluid sampled from the formation contains a target amount or less of contamination.

Aspect 13 is the method of any previous or subsequent aspect, wherein the generating the notification includes communicating the notification to a user device.

Aspect 14 is the method of any previous or subsequent aspect, wherein the numerical model further generates predicted formation properties including one or more of a formation porosity, a formation permeability, a permeability anisotropy, a formation pressure, a formation relative permeability, a formation capillary pressure, a formation water saturation, a formation residual saturation, a formation phase and total mobility, or a formation height.

Aspect 15 is the method of any previous or subsequent aspect, wherein the numerical model evaluates the formation condition by computing a derivative of one or more fluid parameters of the set of fluid parameters.

Aspect 16 is the method of any previous or subsequent aspect, wherein the numerical model evaluates the formation condition by decomposing one or more fluid parameters of the set of fluid parameters as a sum of a plurality of exponential decays.

Aspect 17 is the method of any previous or subsequent aspect, wherein the numerical model applies a noise filter to one or more fluid parameters of the set of fluid parameters.

Aspect 18 is the method of any previous or subsequent aspect, wherein the formation condition is a time at which a fluid contamination level for fluid from the formation falls or is predicted to fall below a threshold level.

Aspect 19 is the method of any previous or subsequent aspect, wherein the set of fluid parameters includes a contamination level for the sampled fluid.

Aspect 20 is the method of any previous, wherein the numerical model evaluates a time at which a fluid contamination level for fluid from the formation falls or is predicted to fall below a threshold level by decomposing measured fluid contamination levels for the sampled fluid as a sum of a plurality of exponentials.

Aspect 21 is a formation testing system, the system comprising a formation testing tool including: one or more sampling systems for obtaining a sampled fluid from a formation; one or more sensors for analyzing the sampled fluid; one or more processors in communication with the one or more sampling systems and the one or more sensors; and a non-transitory computer readable storage medium in communication with the one or more processors, the non-transitory computer readable storage medium containing instructions that, when executed by the one or more processors, cause the one or more processors to perform operations including: using the one or more sampling systems to obtain sampled fluid from the formation according to a set of sampling parameters; using the one or more sensors to analyze the sampled fluid to identify a set of fluid parameters for the sampled fluid; and using the numerical model to determine a formation condition, wherein inputs for the numerical model include the set of sampling parameters and the set of fluid parameters.

Aspect 22 is the system of any previous or subsequent aspect, wherein the operations further include repeating one or more times: using the numerical model to generate an updated set of sampling parameters; using the formation testing tool to obtain additional sampled fluid from the formation according to the updated set of sampling parameters; using the formation testing tool to analyze the additional sampled fluid to identify an updated set of fluid parameters for the additional sampled fluid; and using the numerical model to generate an updated formation condition, wherein inputs for the numerical model further include the updated set of sampling parameters and the updated set of fluid parameters.

Aspect 23 is the system of any previous or subsequent aspect, wherein inputs for the numerical model further include one or more of historical fluid parameters for fluid sampled from the formation, simulated fluid parameters for fluid sampled from the formation, historical fluid parameters for fluid sampled from a different formation, and simulated fluid parameters for fluid sampled from the different formation.

Aspect 24 is the system of any previous or subsequent aspect, wherein the set of sampling parameters comprises sampling conditions associated with obtaining the sampled fluid.

Aspect 25 is the system of any previous or subsequent aspect, wherein the set of sampling parameters comprises a drawdown rate used for sampling fluid from the formation, a drawdown pressure used for sampling fluid from the formation, an injection rate for injecting fluid from the formation testing tool into the formation during sampling, a buildup pressure measured after sealing the testing tool, or a characteristic dimension of the formation testing tool.

Aspect 26 is the system of any previous or subsequent aspect, wherein the set of sampling parameters further comprise a pulse sequence, the pulse sequence including one or more modifications to the drawdown rate, the drawdown pressure, the injection rate, or the buildup pressure in an ordered sequence during sampling fluid from the formation.

Aspect 27 is the system of any previous or subsequent aspect, wherein the set of fluid parameters for the sampled fluid comprises analytical results associated with evaluating the sampled fluid.

Aspect 28 is the system of any previous or subsequent aspect, wherein the set of fluid parameters for the sampled fluid comprises at least one of a mass density for the sampled fluid, a fluid viscosity for the sampled fluid, a fluid resistivity for the sampled fluid, a formation pressure, an estimated formation pressure, an optical density for the sampled fluid, a level of contamination for the sampled fluid, a speed of sound in the sampled fluid, a gas-to-liquid ratio for the sampled fluid, a composition of the sample fluid, or a formation volume factor for the sampled fluid.

Aspect 29 is the system of any previous or subsequent aspect, wherein fluid parameters of the set of fluid parameters are determined as a function of time or a function of pumpout volume.

Aspect 30 is the system of any previous or subsequent aspect, wherein the formation condition comprises one or more of: predicted contamination for additional fluid sampled from the formation as a function of time or pumpout volume; a predicted time at which additional fluid sampled from the formation contains a target amount or less of contamination; a predicted pumpout volume at which additional fluid sampled from the formation contains a target amount or less of contamination; or a predicted lowest level of contamination for additional fluid sampled from the formation.

Aspect 31 is the system of any previous or subsequent aspect, wherein the operations further include: generating a notification providing the formation condition.

Aspect 32 is the system of any previous or subsequent aspect, wherein the notification includes one or more of an indication of a predicted lowest level of contamination for additional fluid sampled from the formation, or a predicted duration until additional fluid sampled from the formation contains a target amount or less of contamination.

Aspect 33 is the system of any previous or subsequent aspect, wherein the generating the notification includes communicating the notification to a user device.

Aspect 34 is the system of any previous or subsequent aspect, wherein the numerical model further generates predicted formation properties including one or more of a formation porosity, a formation permeability, a permeability anisotropy, a formation pressure, a formation relative permeability, a formation capillary pressure, a formation water saturation, a formation residual saturation, a formation phase and total mobility, or a formation height.

Aspect 35 is the system of any previous or subsequent aspect, wherein the numerical model evaluates the formation condition by computing a derivative of one or more fluid parameters of the set of fluid parameters.

Aspect 36 is the system of any previous or subsequent aspect, wherein the numerical model evaluates the formation condition by decomposing one or more fluid parameters of the set of fluid parameters as a sum of a plurality of exponential decays.

Aspect 37 is the system of any previous or subsequent aspect, wherein the numerical model applies a noise filter to one or more fluid parameters of the set of fluid parameters.

Aspect 38 is the system of any previous or subsequent aspect, wherein the formation condition is a time at which a fluid contamination level for fluid from the formation falls or is predicted to fall below a threshold level.

Aspect 39 is the system of any previous or subsequent aspect, wherein the set of fluid parameters includes a contamination level for the sampled fluid.

Aspect 40 is the system of any previous aspect, wherein the numerical model evaluates a time at which a fluid contamination level for fluid from the formation falls or is predicted to fall below a threshold level by decomposing measured fluid contamination levels for the sampled fluid as a sum of a plurality of exponentials.

Aspect 41 is a computer program product comprising a non-transitory computer-readable storage medium storing computer-executable instructions that, when executed by one or more processors, cause the one or more processors to perform operations including: using a formation testing tool to obtain a sampled fluid from a formation according to a set of sampling parameters; using the formation testing tool to analyze the sampled fluid to identify a set of fluid parameters for the sampled fluid; using a numerical model to determine a formation condition, wherein inputs for the numerical model include the set of sampling parameters and the set of fluid parameters.

Aspect 42 is the computer program product of any previous or subsequent aspect, wherein the operations further comprise repeating one or more times: using the numerical model to generate an updated set of sampling parameters; using the formation testing tool to obtain additional sampled fluid from the formation according to the updated set of sampling parameters; using the formation testing tool to analyze the additional sampled fluid to identify an updated set of fluid parameters for the additional sampled fluid; and using the numerical model to generate an updated formation condition, wherein inputs for the numerical model further include the updated set of sampling parameters and the updated set of fluid parameters.

Aspect 43 is the computer program product of any previous or subsequent aspect, wherein inputs for the numerical model further include one or more of historical fluid parameters for fluid sampled from the formation, simulated fluid parameters for fluid sampled from the formation, historical fluid parameters for fluid sampled from a different formation, and simulated fluid parameters for fluid sampled from the different formation.

Aspect 44 is the computer program product of any previous or subsequent aspect, wherein the set of sampling parameters comprises sampling conditions associated with obtaining the sampled fluid.

Aspect 45 is the computer program product of any previous or subsequent aspect, wherein the set of sampling parameters comprises a drawdown rate used for sampling fluid from the formation, a drawdown pressure used for sampling fluid from the formation, an injection rate for injecting fluid from the formation testing tool into the formation during sampling, a buildup pressure measured after sealing the testing tool, or a characteristic dimension of the formation testing tool.

Aspect 46 is the computer program product of any previous or subsequent aspect, wherein the set of sampling parameters further comprise a pulse sequence, the pulse sequence including one or more modifications to the drawdown rate, the drawdown pressure, the injection rate, or the buildup pressure in an ordered sequence during sampling fluid from the formation.

Aspect 47 is the computer program product of any previous or subsequent aspect, wherein the set of fluid parameters for the sampled fluid comprises analytical results associated with evaluating the sampled fluid.

Aspect 48 is the computer program product of any previous or subsequent aspect, wherein the set of fluid parameters for the sampled fluid comprises at least one of a mass density for the sampled fluid, a fluid viscosity for the sampled fluid, a fluid resistivity for the sampled fluid, a formation pressure, an estimated formation pressure, an optical density for the sampled fluid, a level of contamination for the sampled fluid, a speed of sound in the sampled fluid, a gas-to-liquid ratio for the sampled fluid, a composition of the sample fluid, or a formation volume factor for the sampled fluid.

Aspect 49 is the computer program product of any previous or subsequent aspect, wherein fluid parameters of the set of fluid parameters are determined as a function of time or a function of pumpout volume.

Aspect 50 is the computer program product of any previous or subsequent aspect, wherein the formation condition comprises one or more of: predicted contamination for additional fluid sampled from the formation as a function of time or pumpout volume; a predicted time at which additional fluid sampled from the formation contains a target amount or less of contamination; a predicted pumpout volume at which additional fluid sampled from the formation contains a target amount or less of contamination; or a predicted lowest level of contamination for additional fluid sampled from the formation.

Aspect 51 is the computer program product of any previous or subsequent aspect, wherein the operations further include: generating a notification providing the formation condition.

Aspect 52 is the computer program product of any previous or subsequent aspect, wherein the notification includes one or more of an indication of a predicted lowest level of contamination for additional fluid sampled from the formation, or a predicted duration until additional fluid sampled from the formation contains a target amount or less of contamination.

Aspect 53 is the computer program product of any previous or subsequent aspect, wherein the generating the notification includes communicating the notification to a user device.

Aspect 54 is the computer program product of any previous or subsequent aspect, wherein the numerical model further generates predicted formation properties including one or more of a formation porosity, a formation permeability, a permeability anisotropy, a formation pressure, a formation relative permeability, a formation capillary pressure, a formation water saturation, a formation residual saturation, a formation phase and total mobility, or a formation height.

Aspect 55 is the computer program product of any previous or subsequent aspect, wherein the numerical model evaluates the formation condition by computing a derivative of one or more fluid parameters of the set of fluid parameters.

Aspect 56 is the computer program product of any previous or subsequent aspect, wherein the numerical model evaluates the formation condition by decomposing one or more fluid parameters of the set of fluid parameters as a sum of a plurality of exponential decays.

Aspect 57 is the computer program product of any previous or subsequent aspect, wherein the numerical model applies a noise filter to one or more fluid parameters of the set of fluid parameters.

Aspect 58 is the computer program product of any previous or subsequent aspect, wherein the formation condition is a time at which a fluid contamination level for fluid from the formation falls or is predicted to fall below a threshold level.

Aspect 59 is the computer program product of any previous or subsequent aspect, wherein the set of fluid parameters includes a contamination level for the sampled fluid.

Aspect 60 is the computer program product of any previous aspect, wherein the numerical model evaluates a time at which a fluid contamination level for fluid from the formation falls or is predicted to fall below a threshold level by decomposing measured fluid contamination levels for the sampled fluid as a sum of a plurality of exponentials.

### Statements Regarding Incorporation by Reference and Variations

All references throughout this application, for example patent documents including issued or granted patents or equivalents; patent application publications; and non-patent literature documents or other source material; are hereby incorporated by reference herein in their entireties, as though individually incorporated by reference.

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art.

When a group of substituents is disclosed herein, it is understood that all individual members of those groups and all subgroups and classes that can be formed using the substituents are disclosed separately. When a Markush group or other grouping is used herein, all individual members of the group and all combinations and subcombinations possible of the group are intended to be individually included in the disclosure. As used herein, “and/or” means that one, all, or any combination of items in a list separated by “and/or” are included in the list; for example “1, 2 and/or 3” is equivalent to “1, 2, 3, 1 and 2, 1 and 3, 2 and 3, or 1, 2 and 3”.

Every formulation or combination of components described or exemplified can be used to practice the invention, unless otherwise stated. Specific names of materials are intended to be exemplary, as it is known that one of ordinary skill in the art can name the same material differently. It will be appreciate that methods, device elements, starting materials, and synthetic methods other than those specifically exemplified can be employed in the practice of the invention without resort to undue experimentation. All art-known functional equivalents, of any such methods, device elements, starting materials, and synthetic methods are intended to be included in this invention. Whenever a range is given in the specification, for example, a temperature range, a time range, or a composition range, all intermediate ranges and subranges, as well as all individual values included in the ranges given are intended to be included in the disclosure.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element or elements, limitation or limitations which is not specifically disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A method comprising:
using a formation testing tool to obtain a sampled fluid from a formation according to a set of sampling parameters;
using the formation testing tool to analyze the sampled fluid to identify a set of fluid parameters for the sampled fluid; and
using a numerical model to determine a formation condition, wherein inputs for the numerical model include the set of sampling parameters and the set of fluid parameters.

2. The method of claim 1, further comprising repeating one or more times:
using the numerical model to generate an updated set of sampling parameters;
using the formation testing tool to obtain additional sampled fluid from the formation according to the updated set of sampling parameters;
using the formation testing tool to analyze the additional sampled fluid to identify an updated set of fluid parameters for the additional sampled fluid; and
using the numerical model to generate an updated formation condition, wherein inputs for the numerical model further include the updated set of sampling parameters and the updated set of fluid parameters.

3. The method of claim 1, wherein inputs for the numerical model further include one or more of historical fluid parameters for fluid sampled from the formation, simulated fluid parameters for fluid sampled from the formation, historical fluid parameters for fluid sampled from a different formation, and simulated fluid parameters for fluid sampled from the different formation.

4. The method of claim 1, wherein the set of sampling parameters comprises sampling conditions associated with obtaining the sampled fluid.

5. The method of claim 1, wherein the set of sampling parameters comprises a drawdown rate used for sampling fluid from the formation, a drawdown pressure used for sampling fluid from the formation, an injection rate for injecting fluid from the formation testing tool into the formation during sampling, a buildup pressure measured after sealing the testing tool, or a characteristic dimension of the formation testing tool.

6. The method of claim 5, wherein the set of sampling parameters further comprise a pulse sequence, the pulse sequence including one or more modifications to the drawdown rate, the drawdown pressure, the injection rate, or the buildup pressure in an ordered sequence during sampling fluid from the formation.

7. The method of claim 1, wherein the set of fluid parameters for the sampled fluid comprises analytical results associated with evaluating the sampled fluid.

8. The method of claim 1, wherein the set of fluid parameters for the sampled fluid comprises at least one of a mass density for the sampled fluid, a fluid viscosity for the sampled fluid, a fluid resistivity for the sampled fluid, a formation pressure, an estimated formation pressure, an optical density for the sampled fluid, a level of contamination for the sampled fluid, a speed of sound in the sampled fluid, a gas-to-liquid ratio for the sampled fluid, a composition of the sample fluid, or a formation volume factor for the sampled fluid.

9. The method of claim 1, wherein fluid parameters of the set of fluid parameters are determined as a function of time or a function of pumpout volume.

10. The method of claim 1, wherein the formation condition comprises one or more of:
predicted contamination for additional fluid sampled from the formation as a function of time or pumpout volume;
a predicted time at which additional fluid sampled from the formation contains a target amount or less of contamination;
a predicted pumpout volume at which additional fluid sampled from the formation contains a target amount or less of contamination; or
a predicted lowest level of contamination for additional fluid sampled from the formation.

11. The method of claim 1, further comprising:
generating a notification providing the formation condition, wherein the notification includes one or more of an indication of a predicted lowest level of contamination for additional fluid sampled from the formation, or a predicted duration until additional fluid sampled from the formation contains a target amount or less of contamination.

12. (canceled)

13. (canceled)

14. The method of claim 1, wherein the numerical model further generates predicted formation properties including one or more of a formation porosity, a formation permeability, a permeability anisotropy, a formation pressure, a formation relative permeability, a formation capillary pressure, a formation water saturation, a formation residual saturation, a formation phase and total mobility, or a formation height.

15. The method of claim 1, wherein the numerical model evaluates the formation condition by computing a derivative of one or more fluid parameters of the set of fluid parameters.

16. The method of claim 1, wherein the numerical model evaluates the formation condition by decomposing one or more fluid parameters of the set of fluid parameters as a sum of a plurality of exponential decays.

17. The method of claim 1, wherein the numerical model applies a noise filter to one or more fluid parameters of the set of fluid parameters.

18. The method of claim 1, wherein the formation condition is a time at which a fluid contamination level for fluid from the formation falls or is predicted to fall below a threshold level.

19. The method of claim 1, wherein the set of fluid parameters includes a contamination level for the sampled fluid.

20. The method of claim 1, wherein the numerical model evaluates a time at which a fluid contamination level for fluid from the formation falls or is predicted to fall below a threshold level by decomposing measured fluid contamination levels for the sampled fluid as a sum of a plurality of exponentials.

21. A formation testing system, the system comprising
a formation testing tool including:
one or more sampling systems for obtaining a sampled fluid from a formation;
one or more sensors for analyzing the sampled fluid;
one or more processors in communication with the one or more sampling systems and the one or more sensors; and
a non-transitory computer readable storage medium in communication with the one or more processors, the non-transitory computer readable storage medium containing instructions that, when executed by the one or more processors, cause the one or more processors to perform the method of claim 1.

22.-40. (canceled)

41. A computer program product comprising a non-transitory computer-readable storage medium storing computer-executable instructions that, when executed by one or more processors, cause the one or more processors to perform the method of claim 1.

42.-60. (canceled)
